(19) United States
(12) Patent Application Publication
(10) Pub. No.: US 2024/0159675 A1
(43) Pub. Date: May 16, 2024

(54) METHODS AND DEVICES FOR RATIOMETRIC CHARACTERIZATION OF FLUORESCENT PARTICLES

(71) Applicant: NANOTEMPER TECHNOLOGIES GMBH, MUNICH (DE)

(72) Inventors: PHILIPP BAASKE, MUNICH (DE); PATRICK ANDREAS MARKUS LANGER, MUNICH (DE)

(21) Appl. No.: 18/575,347

(22) PCT Filed: Jun. 30, 2022
(86) PCT No.: PCT/EP2022/068114

§ 371 Date: Dec. 29, 2023

(30) Foreign Application Priority Data
Jul. 1, 2021 (EP) 21183266.2

(51) Int. Cl. G01N 21/64

(52) U.S. Cl. CPC G01N 21/6428; G01N 2021/6421

(57) ABSTRACT

The present invention relates to devices and methods for the characterization of fluorescently labeled particles in solution by analyzing alterations in the fluorescence spectrum of the fluorescently labeled particles. In particular, a sample of fluorescently labeled particles is analyzed under different conditions/environments by fluorescent excitation and detection of the corresponding fluorescence emissions. The particles are characterized by analyzing the detected fluorescence emissions under these different conditions/environments. More specifically, the present invention relates to methods and devices for ratiometric characterization of inter- and/or intramolecular interactions, and/or conformational modifications and/or localization of fluorescently labeled particles.

### FIELD OF THE INVENTION

The present invention relates to devices and methods for the characterization of fluorescently labeled particles in solution by analyzing alterations in the fluorescence spectrum of the fluorescently labeled particles. In particular, a sample of fluorescently labeled particles is analyzed under different conditions/environments by fluorescent excitation and detection of the corresponding fluorescence emissions. The particles are characterized by analyzing the detected fluorescence emissions under these different conditions/environments. More specifically, the present invention relates to methods and devices for ratiometric characterization of inter- and/or intramolecular interactions, and/or conformational modifications and/or localization of fluorescently labeled particles.

### BACKGROUND

The fluorescence spectrum of a fluorescent label is sensitive to environmental changes such as changes in the chemical surrounding and temperature changes. Thus, the same fluorescent label can show alterations in its fluorescence spectrum in terms of intensity and/or spectral shifts and/or spectral shape.

Since this effect is well-known, it is used to study interactions of intrinsically or extrinsically fluorescently labeled particles. In the art, alterations in the fluorescence intensity are primarily used to characterize binding reactions by determining dissociation constants (K_{d}) (NPL1, NPL2). Besides, characterizing inter-molecular interactions, the interactions to be determined can further include intra-molecular interactions, and/or modifications (conformational changes) and/or (changes in) localization of fluorescently labeled particles. For example, conformational changes of proteins and analyte concentrations are determined in the art based on methods involving a mechanism called Forster resonance energy transfer (FRET) (WO 2017/087912 A2). The fundamental mechanism of FRET involves a donor fluorophore in an excited electronic state, which may transfer its excitation energy to a nearby acceptor fluorophore through dipole-dipole coupling due to interaction-mediated (e.g., ligand-mediated) changes in the distance and/or the angle between the two fluorophores. As such, FRET measurements require two or more fluorescent labels, i.e. at least one donor and at least one acceptor fluorophore. However, since two different fluorescent labels each of which may have different sensitivities to environmental changes are used, FRET measurements may be falsified by undesired changes in the local environment of the fluorophores. Furthermore, the fluorescence emission from the acceptor fluorophore when being excited via energy transfer usually provides lower signal strength than measurements of acceptor fluorophore emission intensities resulting from direct excitation. Generally, FRET measurements require precise positioning of the donor and acceptor fluorophore(s) within a defined distance on the target molecule, e.g., by applying two site-specific labeling chemistries which, in turn, may not be practicable for every kind of target. In contrast, random labeling, e.g., by lysine-reactive dyes or cysteine-reactive dyes would result in various FRET distances and thus, in failure of the FRET measurements. In summary, in comparison to measurements involving only a single fluorescent label as in the present invention, FRET measurements have a lower signal-to-noise ratio and it is desirable that the fluorescently labeled particles to be analyzed are labeled with only one type of fluorescent label.

Depending on the interaction and the characteristics of the fluorescent label itself, the alterations in the fluorescence spectra can be smaller than 1% and hence, fall in the usual range of pipetting errors. At present, it is difficult if not impossible to resolve alterations smaller than 1% with the methods/devices available in the art.

Alterations in the fluorescence spectrum of a fluorescent label are usually detected with fluorescence spectrophotometers, which are designed to record/measure over a large spectral range and thus, fail to resolve small changes in the fluorescence intensity. Additionally, since commercially available fluorescent labels are presently designed to be more robust against environmental changes, there is a need to tailor super sensitive fluorescent labels in order to increase resolution of spectrophotometry measurements (NPL3, NPL4, NPL5).

Another possibility to enhance the resolution of spectrophotometric measurements can be achieved by combining a plurality of measurements. In this case, the sample to be examined is excited at a first wavelength and its emission is measured at a second wavelength in a first measurement, followed by repeating the excitation step at the first wavelength and measurement of its emission at a third wavelength in a second measurement. The detected emission intensities obtained from the first and second measurement are then combined. Hence, the pure information of interest independent from any artefacts, for example introduced due to manual handling can be determined. However, due to the subsequent implementation of two measurements, this method is more cumbersome and time consuming. Also, the conditions during the first and second measurement may vary. Furthermore, bleaching could have occurred which can further lead to falsification of results. By using this approach, fluorescence changes down to 4.5% were resolved when being measured at room temperature (NPL2).

In general, measurements with commercially available fluorescence spectrophotometric devices require large sample volumes (i.e. by using quartz cuvettes or multi-well plates) and consequently, higher sample concentrations in order to sufficiently resolve alterations in the fluorescence intensity. In the following, further methods known in the art, which require smaller sample volumes (i.e. 10 μl) are discussed.

For example, in the case that the absolute intensity variation at room temperature is too small compared to pipetting errors, pipetting errors can be eliminated by applying a characterization method based on temperature related intensity change (TRIC) (WO 2018/234557). By calculating Fₕₒₜ/F_{cold}, i.e. a ratio of the fluorescence intensity based on the measured intensity at room temperature and the intensity measured at a second, typically higher temperature, meaningful measurements inter- and/or intra-molecular interactions can still be determined.

However, an increase in temperature, even by only a few degrees Celsius (° C.):
is not always tolerated by the particles to be measured. Thus, this method is not suitable to characterize interactions involving unstable samples, such as unstable proteins.
the fluorescence intensity may equally change for the fluorescently labeled particle and for the fluorescently labeled particle complexed with a ligand. In this case, “Fₕₒₜ/F_{cold}” has the same value in both cases and although an interaction between the interaction partners takes place, no binding curve can be obtained.
inhomogeneous samples such as samples containing a certain fraction of aggregates, can lead to irreproducible fluorescence traces due to convection, meaning that the noise in “Fₕₒₜ/F_{cold}” can become substantially large. Thus, it is impossible to obtain binding curves for systems with small signal amplitudes.

Moreover, analyzing interactions of ternary complexes, such as complexes containing a labeled molecule A, another molecule B that binds to A and a third molecule C that binds to B, the change in TRIC is often not sufficient to resolve said interactions.

In cases where the interaction changes rapidly with temperature and binding of the ligand causes fluorescence intensity changes, the TRIC method can result in either biphasic dose-response-curves, which cannot be analyzed with a sigmoidal 1-to-1 binding model, or the measured value of the dissociation constant may deviate from the actual value (e.g. the measured binding affinity corresponds to the weaker binding at the higher temperature instead of the lower temperature).

A different approach to investigate intra- and/or inter- molecular interactions of fluorescent particles is nano differential scanning fluorimetry (WO 2017/055583). This method is based on measuring changes of the intrinsic fluorescence intensity of proteins containing Tryptophan (Trp) and Tyrosine (Tyr) residues. However, proteins typically contain several of those fluorescent aromatic amino acid residues. Since usually not all of those are involved in a binding reaction, upon excitation a high fluorescence background occurs which decreases the amplitude of the signal. Moreover, Trp and Tyr residues are typically located in the hydrophobic core of a protein and may not be affected much by binding of a ligand. Since often lying in the same range as the fluorescence of Trp and Tyr, the auto-fluorescence of ligands interferes with the readout.

In contrast, by labeling with an extrinsic fluorescence label it can be controlled that only one dye (i.e. one type of dye) is attached to a target molecule and only that dye needs to be affected by ligand binding. Furthermore, the labeling chemistry can be tailored so that the dye can be placed in a location that is optimal for detecting changes in the chemical micro-environment (e.g. in the proximity of the ligand binding site). Moreover, extrinsic dyes are usually located on the protein surface and therefore have an ideal exposure to sense changes to the chemical micro-environment. The dye fluorescence range can be chosen so that it does not interfere with auto-fluorescence of a ligand. Lastly, extrinsic dyes are much brighter, and measurements can occur at much lower concentration of the target molecule, thus reducing sample consumption and allowing the measurement of even picomolar affinities.

Thus, there is a need for improved or alternative methods or improved or alternative devices for characterization of inter- and/or intramolecular interactions, and/or modifications (conformational changes) and/or (changes in) localization of fluorescently labeled particles.

### SUMMARY OF THE INVENTION

The present invention provides new methods and devices for characterization of inter- and/or intramolecular interactions, and/or modifications (conformational changes) and/or (changes in) localization of fluorescently labeled particles as defined by the features of the independent claims. Further preferred embodiments of the present invention are defined in the dependent claims. In particular, the present invention solves the technical problem of economically resolving arbitrarily small alterations in the fluorescence spectrum of labeled fluorescent particles based on preferably exactly one fluorescent label and independent from the characteristics (e.g. temperature stability, aggregate formation) and size of the fluorescently labeled particle.

The present invention relates to methods and devices for ratiometric characterization of inter- and/or intramolecular interactions, and/or conformational modifications and/or localization of fluorescently labeled particles. In particular, the methods of the present invention provide an improved sensitivity in detecting alterations in the fluorescence spectrum of fluorescent labels within small sample volumes of fluorescently labeled particles, which could not have been resolved by previously known methods. Further, the method of the present invention enables fast measurements of even temperature sensitive and/or unstable samples. In combination with a defined temperature perturbation, the method of the present invention enables the measurements of thermodynamic and kinetic parameters of interactions. In the methods of the present invention, fluorescently labeled particles are employed which are preferably labeled with at least one fluorescent label. The method of the present invention enables to determine the localization of fluorescently labeled particles (e.g. if the fluorescently labeled particle is located within lipid nanoparticles (LNPs) and/or within cells and/or within a buffer solution surrounding the LNPs and cells).

In a first aspect, the present invention relates to a method for the characterization of fluorescently labeled particles in solution by analyzing alterations in the fluorescence spectrum of the fluorescently labeled particles. The method of the first aspect comprises the following steps:

a) providing a sample of the fluorescently labeled particles in a solution under first conditions,

b) exciting the fluorescently labeled particles at a first wavelength,

c) detecting the fluorescence emission intensity of the fluorescently labeled particles at a second and a third wavelength,

d) calculating the ratio between said fluorescence intensities at the second and the third wavelength,

wherein said third wavelength is different from said second wavelength. It is further preferred to

e1) repeat steps b) to d) for said sample of the fluorescently labeled particles under second conditions, or to

e2) repeat the steps a) to d) for a second sample of the fluorescently labeled particles under second conditions,

wherein said second conditions are different from the said first conditions.

f) The fluorescently labeled particles can then be characterized based on the calculated ratios obtained for the different conditions,

wherein the second and third wavelength are preferably detected simultaneously, and wherein the second wavelength is preferably shorter and the third wavelength is preferably longer than an emission maximum of the fluorescence emission of the fluorescently labeled particles under the first conditions.

In a second aspect, the present invention relates to a method for the characterization of fluorescently labeled particles in solution by analyzing alterations in the fluorescence spectrum of the fluorescently labeled particles in combination with a defined temperature perturbation/temperature change. The method of the second aspect comprises the following steps:

a) providing a sample of the fluorescently labeled particles in a solution under first conditions,

b) exciting the fluorescently labeled particles at a first wavelength,

c) detecting the fluorescence emission intensity of the fluorescently labeled particles at a second and a third wavelength,

wherein said intensities are detected during a defined temperature perturbation,

d) calculating the ratio between said fluorescence intensities at the second and the third wavelength, wherein said third wavelength is different from said second wavelength.

Like for the first aspect, it is also preferred,

e1) to repeat steps b) to d) for said sample of the fluorescently labeled particles under second conditions, or

e2) to repeat the steps a) to d) for a second sample of the fluorescently labeled particles under second conditions,

wherein said second conditions are different from the said first conditions, and

f) characterizing the fluorescently labeled particles based on the calculated ratios obtained for the different conditions,

wherein the second and third wavelength are preferably detected simultaneously, and wherein the second wavelength is preferably shorter and the third wavelength is longer than an emission maximum of the fluorescence emission of the fluorescently labeled particles under the first conditions.

In a third aspect, the present invention relates to a method for the characterization of the thermodynamic and/or kinetic parameters of fluorescently labeled particles in solution by analyzing alterations in the fluorescence spectrum of the fluorescently labeled particles in combination with a defined temperature perturbation/temperature change.

In a fourth aspect, the present invention relates to a method for the characterization of the localization of fluorescently labeled particles in solution by analyzing alterations in the fluorescence spectrum of the fluorescently labeled particles in combination with or without a defined temperature perturbation/temperature change.

It is preferred in the context of the present invention, that the fluorescence emission intensities at a second and a third wavelength of step c) are detected simultaneously, which preferably means within a short time interval of less than 1 s, more preferably less than 750 ms, more preferably less than 500 ms, more preferably less than 250 ms, more preferably less than 100 ms, more preferably less than 50 ms, more preferably less than 25 ms, more preferably less than 10 ms, more preferably less than 5 ms, even more preferably less than 2.5 ms. In this context, typical time intervals are 50 ms,10 ms and 1 ms.

Some particular aspects of the invention can be summarized as follows:

In some aspects the methods described herein comprise the steps of a) providing a sample of the fluorescently labeled particles in a solution under first conditions, b) exciting the fluorescently labeled particles at a first wavelength, c) detecting the fluorescence emission intensity of the fluorescently labeled particles at a second and a third wavelength, d) calculating the ratio between said fluorescence intensities at the second and the third wavelength, wherein said third wavelength is different from said second wavelength, e1) repeating steps b) to d) for said sample of the fluorescently labeled particles under second conditions, or e2)repeating the steps a) to d) for a second sample of the fluorescently labeled particles under second conditions, wherein said second conditions are different from said first conditions, f) characterizing the fluorescently labeled particles based on the calculated ratios obtained for the different conditions, wherein the second and third wavelength are detected simultaneously, and wherein the second wavelength is shorter, and the third wavelength is longer than an emission maximum of the fluorescence emission of the fluorescently labeled particles under the first conditions.

In some aspects of the present invention, the sample volume containing the fluorescently labeled particles is less than 100 μl, preferably between 1 μl and 25 μl, i.e., the sample containing the fluorescently labeled particles is provided in a volume of less than 100 μl, preferably between 1 μl and 25 μl.

It is preferred that in some aspects of the present invention, the sample containing the fluorescently labeled particles is provided in a volume between 1 μl and 25 μl.

In some aspects of the present invention, the sample containing the fluorescently labeled particles is provided in a capillary.

In some aspects of the present invention, the fluorescently labeled particles are labeled with an environment sensitive label.

In some aspects of the present invention, the particles are selected from the group consisting of organic molecules, biomolecules, nanoparticles, microparticles, vesicles, biological cells or sub-cellular fragments, biological tissues, viral particles, viruses, cellular organelles, lipid nanoparticles (LNPs), and virus like particles.

In some aspects of the present invention, the biomolecules are selected from the group consisting of amino acids, proteins, peptides, mono- and disaccharides, polysaccharides, lipids, glycolipids, fatty acids, sterols, vitamins, neurotransmitter, enzymes, nucleotides, metabolites, nucleic acids, and combinations thereof.

In some aspects of the present invention, the concentration of the fluorescently labeled particles in the solution is from 10 pM to 10 μM, preferably 50 pM to 500 nM.

In some aspects of the present invention, the alterations in the detected fluorescence intensity of the fluorescently labeled particles result from spectral shifts or broadening of the spectrum or narrowing of the spectrum, or combinations thereof.

In some aspects of the present invention, the fluorescence intensity of the fluorescently labeled particles changes due to mechanisms selected from the group consisting of conformational changes of the fluorescently labeled particles, re-localization of the fluorescently labeled particles, interactions between the fluorescently labeled particles and one or more ligands, and combinations thereof.

In some aspects of the present invention, the calculated ratios obtained in step f) are used to determine the localization of the fluorescently labeled particles or parameters selected from the group consisting of dissociation constants, half maximal effective concentrations (EC₅₀), equilibrium constants, binding kinetics, enzymatic reaction kinetics, thermodynamic parameters, unfolding or refolding kinetics, opening and closing reactions, and combinations thereof.

In some aspects of the present invention, the second conditions of step e) are altered by adding a ligand and/or different concentrations of the ligand and the calculated ratios obtained in step f) are used to determine the dissociation constant of the fluorescently labeled particles and the ligand.

In some aspects of the present invention, the first and second conditions of the fluorescently labeled particles differ with regard to their temperature and/or chemical composition.

In some aspects of the present invention, the fluorescence emission intensity of the fluorescently labeled particles at a second and a third wavelength of step c) is detected during a defined temperature perturbation.

Also provided herein is a device for the characterization of fluorescently labeled particles in solution by analyzing alterations in the fluorescence spectrum of the fluorescently labeled particles. In some aspects, the device of the present invention is adapted for performing the methods described herein. In some aspects the device comprises a sample holder for holding a sample of fluorescently labeled particles in solution under a plurality of conditions (i.e. multiple different conditions); means for exciting the fluorescently labeled particles at a first wavelength; means for detecting the fluorescence emission intensity of the fluorescently labeled particles at a second and a third wavelength; means for calculating a ratio between said fluorescence intensities at the second and the third wavelength, wherein said third wavelength is different from said second wavelength; wherein the device is configured to consecutively excite fluorescently, detect fluorescence emissions and calculate the ratio for samples at different conditions; means for characterizing the fluorescently labeled particles based on the calculated ratios obtained for the different conditions, wherein device is configured to simultaneously detect the second and third wavelength, and wherein the second wavelength is shorter and the third wavelength is longer than an emission maximum of the fluorescence emission of the fluorescently labeled particles under a first condition (of the different conditions).

In some aspects of the present invention, the means for exciting is an excitation light source, preferably at least one light source from the group consisting of laser, laser fibre laser, diode-laser, LED, HXP, Halogen, LED-Array, HBO.

In some aspects of the present invention, the means for detecting is light detector, preferably at least one detector from the group consisting of PMT, siPM, APD, CCD or CMOS camera.

Also provided herein is a computer program comprising instructions, which when the program is executed by a computer, cause the computer is used to carry out the methods described herein

Also provided herein is a computer-readable data carrier comprising instructions, which when executed by a computer, cause the computer is used to carry out the method described herein

Also provided herein is the use of a device for the characterization of fluorescently labeled particles in solution according to the methods described herein.

Also provided herein is the use of a capillary for the characterization of fluorescently labeled particles in solution by analyzing alterations in the fluorescence spectrum of the fluorescently labeled particles, wherein a sample of the fluorescently labeled particles in a solution is filled in the capillary and provided for analyzing, according to the methods described herein.

### BRIEF DESCRIPTION OF THE FIGURES

In the following, preferred embodiments of the present invention are described in detail with respect to the Figures.

FIG. 1 (A) shows the excitation spectra of four different protein samples labeled with the identical fluorescent dye. The samples were excited between 520 and 670 nm. The emission of the samples was recorded at 690 nm. (B) Zoom-in image into the excitation peaks of (A).

FIG. 2 (A) shows the emission spectra of four different protein samples labeled with the identical fluorescent dye. The samples were excited at 605 nm. The emission of the samples was recorded between 620 and 750 nm. (B) Zoom-in image into the emission peaks of (A).

FIG. 3 shows potential effects of (A) ligand proximity and (B) conformational changes of a labeled molecule on alterations of the fluorescence spectrum of a fluorescent dye including (C) hypsochromic (blue) or bathochromic (red) shifts and/or (D) broadening or narrowing of the spectrum.

FIG. 4 (A) shows the emission peaks of the fluorescently labeled protein streptavidin alone and in combination with its natural ligand biotin. (B) Zoom-in image into the emission peaks of (A).

FIG. 5 (A) shows the emission peaks of the fluorescently labeled protein lysozyme alone and in combination with the inhibitor Tri-N-acetyl-D-glucosamine (NAG3). (B) Zoom-in image into the emission peaks of (A).

FIG. 6 (A) shows the ratio traces of Carbonic anhydrase in complex with furosemide detected by using the dual-emission configuration of the present invention. The change in the fluorescence ratio of Carbonic anhydrase upon furosemide binding is 0.5%. (B) shows the resulting dose-response curve of the binding interaction.

FIG. 7 (A) shows the ratio 350 nm/330 nm of intrinsic tryptophan fluorescence of unlabeled lysozyme and different concentrations of NAG3 during increasing temperatures. To monitor the temperature of denaturation, each sample is heated from 35° C. to 95° C. Increasing concentrations of NAG3 lead to thermal stabilization, i.e. a thermal shift, of lysozyme. This shift cannot be used to extract the dissociation constant of this interaction. (B) By plotting the initial ratio 350 nm/330 nm of the intrinsic protein fluorescence at 35° C. against the concentration of NAG3, a sigmoidal dose-response curve and hence, the dissociation constant (K_{d}) at said temperature is obtained.

FIG. 8 shows different embodiments according to the present invention. (A) Preferred embodiment with the dual-emission optics and the IR laser. (B) Embodiment with the dual-emission optics. (C) Embodiment with the dual-excitation optics and the IR laser. (D) Embodiment with dual-excitation optics. (E) Embodiment with the dual-excitation as well as dual-emission optics and the IR laser. (F) Embodiment with the dual-excitation as well as dual-emission optics.

FIG. 9 shows exemplary filter configurations applicable for the (A) dual-excitation and the (B) dual-emission configuration for red (Cy5) and green (Cy3) fluorescent dyes, respectively.

FIG. 10 shows dose-response curves between Cy5-labled DNA aptamer and AMP obtained by ratiometric characterization based on either (A) a measurement with a commercially available microplate-reader or (B & C) a measurement with the dual-emission configuration according to the present invention.

FIG. 11 shows the fluorescence traces of a Cy5-labeled DNA aptamer mixed with a dilution series of AMP. At timepoint 0 s, an IR laser is switched on and the response of the fluorescence intensity is measured over a period of 31 s. In the present dual-emission configuration, the emitted fluorescence traces were simultaneously recorded (A) at a wavelength of 628 to 653 nm (“650 nm”) and (B) at a wavelength of 665 to 727 nm (“670 nm”).

FIG. 12 shows the analysis of the initial fluorescence intensities of the measurement of FIG. 9. Based on the initial fluorescence intensities obtained for (A) 650 nm and (B) 670 nm, no sigmoidal dose-response curve and hence, no affinities of the interaction are obtained.

FIG. 13 (A) shows the ratiometric analysis of the fluorescence intensity traces of the measurements of FIG. 11, which are obtained by pointwise division of the fluorescence traces at 670 nm by the fluorescence traces at 650 nm. Three different phases i.e. Phase 1 to Phase 3, of the measurement are highlighted. (B) By analyzing the ratio during Phase 1, i.e. before the IR laser is turned on, a dose-response curve with a signal-to-noise ratio greater than 300 is obtained.

FIG. 14(A) shows a K_{d}-over-time-curve for the ratiometric data of FIG. 13A. “Vertical slices” taken in 200 ms intervals are analyzed to obtain a dose-response curve for each of the time intervals. When the temperature change over time is known and the interaction is equilibrating on a faster timescale than the temperature change occurs, a K_{d}-over-temperature relationship can be obtained. (B) By performing Van't Hoff analysis, the binding enthalpy (ΔH) and the binding entropy (ΔS) of the interaction can be determined from said K_{d}-over-temperature relationship.

FIG. 15 shows the fluorescence traces of a Cy3-labeled DNA aptamer mixed with a dilution series of AMP. At timepoint 0 s, an IR laser is switched on and the response of the fluorescence intensity is measured over a period of 6 s. In the present dual-excitation configuration, the emitted fluorescence traces are subsequently recorded by a single detector upon (A) a first excitation with a blue LED at a wavelength of 475 to 495 nm and (B) a subsequent excitation with a green LED at a wavelength of 550 to 575 nm.

FIG. 16(A) shows the ratiometric analysis of the initial fluorescence intensities of the measurement of FIG. 15, which are obtained by pointwise division of the fluorescence traces measured upon excitation with the green LED by the ones measured upon excitation with the blue LED. Three different phases i.e. Phase 1 to Phase 3, of the measurement are highlighted. (B) By analyzing the ratio during Phase 1, i.e. before the IR laser is turned on, a dose-response curve with a signal-to-noise ratio of approx. 80 is obtained. (C) By analyzing the ratio during Phase 3, i.e. after the IR laser is turned on, a dose-response curve with an improved signal-to-noise ratio greater than 130 is obtained.

FIG. 17 shows the dose response curve of a 12-point dilution series of biotin mixed with fluorescently labeled streptavidin obtained by the ratiometric measurement with the dual-emission configuration. The dashed line is a 1-to-1 -binding model fit. Since the target concentration is much higher than the dissociation constant (K_{d}), a characteristic kink at the stoichiometry point can be observed.

FIG. 18 shows the dose response curve of a 15-point dilution series of the small molecule acetazolamide mixed with fluorescently labeled bovine carbonic anhydrase II obtained by the ratiometric measurement with the dual-emission configuration. The dashed line is a 1-to-1-binding model fit. The ratio changes only by approx. 0.7%.

FIG. 19 (A) shows the dose response curve of a 16-point dilution series of unlabeled monoclonal antibody Herceptin (Trastuzumab) mixed with (B) a preformed complex of biotinylated protein L and fluorescently labeled monovalent streptavidin, thus resulting in a ternary complex, obtained by the ratiometric measurement with the dual-emission configuration.

FIG. 20(A) shows a schematic representation of a complex of maltose, biotinylated maltose binding protein, streptavidin and fluorescently labeled biotinylated DNA. (B) shows the dose-response curve between biotinylated maltose binding protein, fluorescently labeled by mixing with unlabeled streptavidin and fluorescently labeled biotinylated DNA, and maltose obtained by the ratiometric measurement with the dual-emission configuration.

FIG. 21 shows the dose response curve of a 14-point dilution series of Angiotensin-converting enzyme 2 (ACE2) mixed with 20 nM Cov-19 Spike protein that was labeled by adding 5 nM of the fluorescently labeled therapeutic antibody CR3022.

FIG. 22 shows four subsequent measurements of the fluorescence ratio of fluorescently labeled Mitogen-activated protein kinase 14 (p38-α) over a time period of about 20 minutes. The fluorescence ratio is not constant for the four measurements, but seems to increase linearly, which indicates that the protein is not stable at room temperature but gradually denatures.

FIG. 23 shows K_{d}-over-time curves for the interaction between (A) Cy5-labeled DNA aptamer for adenosine and the small molecule AMP. (B & C) The DNA hybridization between two 11-mer complementary DNA strands where one strand was labeled with Cy5 measured at (B) 32° C. and (C) 22° C. How quickly the K_{d}-over-time curve can follow the temperature perturbation of the IR laser indicates how fast the binding and dissociation kinetics of that interaction are.

FIG. 24 (A) shows normalized K_{d}-over-time curves for the DNA hybridization between 22° C. and 32° C. The y-axis indicates the fold increase of Kd throughout the measurement (all normalized to 1 for comparison). The x-axis indicates the on time of the IR laser. (B) shows a K_{d}-over-time curve for a measurement of the DNA hybridization with the dual-emission configuration with IR laser according to the present invention. The sample temperature was 22° C. and the temperature after IR laser heating was approx. 32° C. The K_{d} value changes from approx. 10 nM to approx. 500 nM during the IR laser on-time. (C) shows the results of a van't Hoff analysis of the two K_{d} values at the two different temperatures. (D & E) show the interaction's thermodynamic parameters obtained from a classical van't Hoff analysis of the fluorescence ratio measurements at six different sample temperatures (22° C., 24° C., 26° C., 28° C., 30° C., 32° C.) which yields very similar thermodynamic parameters but takes longer than the thermodynamics measurement with IR laser.

FIG. 25 shows simulated K_{d}-over-time curves for different dissociation rates. The legend indicates the different off-rates used in the simulation. (A) shows that the dissociation rates between 10 s⁻¹ and 0.001 s⁻¹ can be resolved with a typical measurement that involved 20 s of IR laser heating. (B) reveals that even small differences between 0.036 s⁻¹ and 0.154 s⁻¹ can be well resolved.

FIG. 26 shows measurements of slow binding kinetics using ratiometric fluorescence signals for a mix-and-measure approach of fluorescently labeled nanobody, which is rapidly mixed with six different concentrations of Cov-19 spike RBD. 2 nM of fluorescently labeled nanobody is mixed rapidly with six different concentrations of COV-19 spike RBD (20 nM-625 pM). Next, ratiometric fluorescence measurements are performed every 90 s to follow the slow binding kinetics. A global fitting model can yield the kₒₙ, k_{off} and K_{d} of the interaction.

FIG. 27(A) shows schematic representations of fluorescently labeled mRNA, a lipid nanoparticle (LNP) and a cell. The ratiometric measurement can be used to determine the localization of the fluorescently labeled mRNA molecules. (B) shows that all fluorescently labeled mRNA molecules are located within an LNP. (C) shows that all fluorescently labeled mRNA molecules are located in the buffer containing chamber. (D) shows that all fluorescently labeled mRNA molecules are located within a cell. (E) shows the evenly distribution of the labeled mRNA molecules in the LNP, the buffer chamber and the cell.

FIG. 28 (A) shows ratiometric measurements of LNPs loaded with fluorescently labeled mRNA in different states. (B) Additional information of the state of the LNPs can be obtained by analyzing the fluorescence traces at a single wavelength (here: 670 nm) which reveal “bumpy” aggregation traces as obtained after a 20 min centrifugation step or “smooth” traces as obtained after heating to 90° C. for 10 min.

FIG. 29 (A) shows a schematic representation of a complex between a tetrameric streptavidin, two biotinylated fluorescently labeled linker molecules and a biotinylated target molecule. The ratiometric measurement in the dual-emission configuration can be used to determine (B) the stoichiometry of a complex between tetrameric streptavidin and biotinylated fluorescently labeled linker molecules and (C) the dose-response curve between the streptavidin-linker complex and a biotinylated target molecule.

### DETAILED DESCRIPTION OF THE INVENTION

The present invention provides methods and devices for ratiometric characterization of inter- and/or intramolecular interactions, and/or conformational modifications and/or localization of fluorescently labeled particles. In particular, the methods of the present invention provide an improved sensitivity in detecting alterations in the fluorescence spectrum of fluorescent labels within small sample volumes of fluorescently labeled particles, which could not have been resolved by previously known methods (e.g. see Example 1). Further, the method of the present invention does not necessarily rely on temperature induced fluorescence spectrum changes and hence, enables fast measurements of even temperature sensitive and/or unstable samples. In combination with a defined temperature perturbation, the method of the present invention enables the measurements of thermodynamic and kinetic parameters of interactions.

The method of the present invention enables to determine the localization of fluorescently labeled particles (i.e. if the fluorescently labeled particle is located within lipid nanoparticles (LNPs) and/or within cells and/or within a buffer solution surrounding the LNPs and cells). In the methods of the present invention, fluorescently labeled particles are employed which are labeled with only one fluorescent label. “Labeled with only one fluorescent label” herein means “labeled with only one type of fluorescent label”. This in turn can be a labelling with only one single fluorescent moiety (e.g. one Cy5 molecule), or two or more fluorescent moieties of only a single type (e.g. two or more Cy5 molecules).

In the context of the present invention alterations in the fluorescence spectrum of fluorescently labeled particles are measured and ratiometrically analyzed in order to characterize interactions, including binding affinities and the like of said fluorescently labeled particles.

The steps of the method of the present invention comprise providing one or more samples of fluorescently labeled particles in solution. The one or more samples are preferably fluorescently excited at a constant excitation wavelength. The emitted fluorescence is preferably simultaneously detected at two different emission wavelengths, preferably at a predetermined constant temperature. The ratio of the fluorescence intensity at the two emissions wavelengths can be determined. Thus, the two obtained emitted fluorescence measurements can be characterized in a ratiometric way.

Since the detection of the two emission wavelengths is preferably performed simultaneously and hence, identical disturbance values or errors impact the measurements, the ratiometric characterization leads to the extraction of pure information. and thus, improves the resolution of the measurement method.

The present inventors have found that the alterations in the fluorescence spectrum of a fluorescent label bound to a particle such as a biomolecule can be used to determine, inter alia the conformational state (folded/unfolded state) and/or interaction parameters between a ligand and a biomolecule.

In the context of the present invention, the terms “detected” and “recorded” are used interchangeable and refer to the determination of the fluorescence signal of fluorescently labeled particles.

In a first aspect, the present invention relates to a method for the characterization of fluorescently labeled particles in solution by analyzing alterations in the fluorescence spectrum of the fluorescently labeled particles at e.g. a predetermined temperature.

The method of the first aspect of the invention comprises the following steps:

a) providing a sample of the fluorescently labeled particles in a solution under first conditions,

b) exciting the fluorescently labeled particles at a first wavelength,

c) detecting the fluorescence emission intensity of the fluorescently labeled particles at a second and a third wavelength,

d) calculating the ratio between said fluorescence intensities at the second and the third wavelength, wherein said third wavelength is different from said second wavelength,

e1) repeating steps b) to d) for said sample of the fluorescently labeled particles under second conditions, or

e2) repeating the steps a) to d) for a second sample of the fluorescently labeled particles under second conditions,

wherein said second conditions are different from the said first conditions,

f) characterizing the fluorescently labeled particles based on the calculated ratios obtained for the different conditions,

wherein the second and third wavelength are detected simultaneously, and wherein the second wavelength is shorter and the third wavelength is longer than an emission maximum of the fluorescence emission of the fluorescently labeled particles under the first conditions.

### Particles

According to the present invention the term “particles” includes molecules, in particular organic molecules, biomolecules, nanoparticles, microparticles and vesicles. The application of the present invention to biomolecules such as nucleic acids and proteins is particularly important. The term “particles” also includes biological cells (e.g., bacterial or eukaryotic cells) or sub-cellular fragments, biological tissues, viral particles, virus-like particles or viruses and cellular organelles, lipid nanoparticles (LNPs) and the like. Nanoparticles also include nanodiscs. A nanodisc is a synthetic model membrane system composed of a lipid bilayer of phospholipids with the hydrophobic edge screened by two amphipathic proteins.

Biomolecules are preferably selected from the group consisting of amino acids, proteins, peptides, mono- and disaccharides, polysaccharides, lipids, glycolipids, fatty acids, sterols, vitamins, neurotransmitter, enzymes, nucleotides, metabolites, nucleic acids, and combinations or complexes thereof. More preferably, the biomolecules are selected from the group consisting of proteins, peptides, enzymes, nucleic acids, and combinations or complexes thereof.

Preferably, the particles (in the labeled particles) are biomolecules, most preferably proteins or nucleic acids.

The proteins are selected from the group consisting of enzymes (e.g., carbonic anhydrase, beta lactamase TEM1, or kinases such as MEK1 and p38), transporter proteins (e.g., MBP), inhibitory proteins (e.g., beta lactamase inhibitory protein BLIP, Anakinra), structural proteins, signaling proteins, ligand-binding proteins, chaperones (e.g., heat shock protein HSP90), antibodies (e.g., Trastuzumab), membrane proteins, and receptors (e.g., interleukin 1 receptor).

Nucleic acids include DNA, RNA (e.g. mRNA, tRNA, rRNA and the like), LNA and PNA. Also, modified (e.g. chemically modified) nucleic acids can be analyzed in the context of the present invention. Locked nucleic acid (LNA), often referred to as inaccessible RNA, is a modified RNA nucleotide. The ribose moiety of an LNA nucleotide is modified with an extra bridge connecting the 2′ oxygen and 4′ carbon. Peptide nucleic acid (PNA) is an artificially synthesized polymer similar to DNA or RNA. DNA and RNA have a deoxyribose and ribose sugar backbone, respectively, whereas PNA's backbone is composed of a peptide such as repeating N-(2-aminoethyl)-glycine units linked by peptide bonds. The various purine and pyrimidine bases are linked to the backbone by a methylene bridge (—CH₂—) and a carbonyl group (—(C═O)—).

In the context of the present invention, a nanoparticle is a particle having an average size of less than 100 nm. The term “average size” describes the mean effective diameter as measured by dynamic light scattering using, for example, Brookhaven Instruments' 90Plus or Malvern Zetasizer Z90 particle sizing instrument. Preferably, the nanoparticle size is in the range of 1 nm to 100 nm, preferably 1 to 70 nm. The nanoparticles can be organic or inorganic particles. The nanoparticles can also be present as composite particles, such as an inorganic core having organic molecules attached to its surface.

A microparticle is a microscopic particle which has a longest dimension of less than 1 mm but normally more than 100 nm. Sizing methods employing transmission electron microscopy (TEM), scanning electron microscopy (SEM), and quasi-elastic light scattering (QELS) may be used to characterize the microparticle. The microparticles can also be present in the form of microbeads.

The microparticles can be, e.g., coated or uncoated silica-/glass-/biodegradable particles, polystyrene-/coated-/flow cytometry-/PMMA-/melamine-/NIST particles, agarose particles, magnetic particles, coated or uncoated gold particles or silver particles or other metal particles, transition metal particles, biological materials, semiconductors, organic and inorganic particles, fluorescent polystyrene microspheres, non-fluorescent polystyrene microspheres, composite materials, liposomes, cells and the like.

Commercially available microparticles are available in a wide variety of materials, including ceramics, glass, polymers, and metals. Microparticles encountered in daily life include pollen, sand, dust, flour, and powdered sugar. In biological systems, microparticles are small membrane bound vesicles circulating in the blood derived from cells that are in contact with the bloodstream, such as platelets and endothelial cells.

Microbeads are preferably manufactured solid plastic particles of less than 5 mm in their largest dimension. Microbeads may also be uniform polymer particles, typically 0.5 to 500 μm in diameter.

The term “modified particle” or “modified bead” relates, in particular, to beads or particles which comprise or are linked to molecules, preferably biomolecules. This also comprises the coating of such beads or particles with these (bio)molecules.

Particles or beads according to this invention may be modified in such a way that, for example, biomolecules, e.g., DNA, RNA or proteins, may be able to bind (in some embodiments specifically and/or covalently) to the particles or beads. Therefore, within the scope of this invention is the analysis of characteristics of beads and/or particles and in particular of molecules attached to or linked to such beads or particles. In particular, such molecules are biomolecules. Accordingly, the term “modified (micro)beads/(nano- or micro)particles”, in particular, relates to beads or particles which comprise additional molecules to be analyzed or characterized. Modified or non-modified microparticles/(nano- or micro)particles may be able to interact with other particles/molecules such as biomolecules (e.g., DNA, RNA or proteins) in solution.

The preferred concentration of the fluorescently labeled particles used in the present invention is preferably from 10 pM to 10 μM, even more preferably from 50 pM to 500 nM.

It is preferred that in the methods of the present invention, the concentration of the fluorescently labeled particles in the solution is from 50 pM to 500 nM.

In the methods of the present invention, labeled particles are employed which are labeled with at least one fluorescent label, preferably exactly one fluorescent label. In the context of this invention, particles labeled with more than one fluorescent label are labeled with only one type of fluorescent label (i.e. only a single type of label per particle). In the context of this invention, “labeled particles” refer to fluorescently labeled particles or other particles which can be detected by fluorescence means, e.g., molecules/particles comprising an intrinsic fluorophore, or particles/molecules tagged with fusion proteins or particles/molecules with extrinsic fluorophores attached.

In particular, the labeled particles are preferably particles which are attached, e.g., covalently bonded to a label (e.g. via NHAs labeling, maleimide labeling and the like), reversibly bonded to a label over a high affinity protein tag, e.g. HIS-tag, AVI-tag, SPOT-tag, SNAP-tag and the like or bioconjugated via Copper(I)-catalyzed azide-alkyne cycloaddition (CuAAC), Strain-promoted azide-alkyne cycloaddition (SPAAC) and the like (also known as click-chemistry) or similar.

Protein tags are peptide sequences genetically grafted onto a recombinant protein. These include poly(His) tag, polyanionic amino acids, such as FLAG-tag, epitope tags like V5-tag, Myc-tag, HA-tag and NE-tag, tags that may allow specific enzymatic modification (such as biotinylation by biotin ligase) or chemical modification (such as reaction with FlAsH-EDT2 for fluorescence imaging).

### Fluorescent Labels

In the context of the present invention the terms “label” and “dye” are used interchangeably and refer to a fluorophore/fluorochrome, i.e., a fluorescent chemical compound, which is re-emitting light upon excitation.

Labels useful in the present invention are labels which are sensitive to environmental changes, i.e., the fluorescence spectrum of the dye alters upon environmental changes such as changes in the chemical microenvironment (ligand binding, conformational changes) and/or macroenvironment (e.g., location in LNPs versus location in cells) and/or temperature changes (e.g., heating or cooling).

In the context of the present invention the fluorescent label is advantageously attached to the particle in proximity to a location/position on said particle where a binding interaction is expected to take place, e.g., the binding pocket of a protein or the like.

Fluorescent labels for use according to the present invention can be selected from the group consisting of intrinsic fluorescent labels, fusion proteins, extrinsic fluorescent labels or the like.

Intrinsic fluorescent labels include tryptophan residues, tyrosine residues, phenylalanine residues. Fusion proteins can be selected from the group consisting of blue-emitting fluorescent proteins, cyan-emitting fluorescent proteins, green-emitting fluorescent proteins, yellow-emitting fluorescent proteins and red-emitting fluorescent proteins and the like. Reference is made to FPbase a well-known data base in the art, which provides a comprehensive list of presently known fluorescent proteins (https://www.fpbase.org/table/; Lambert, T J (2019) FPbase: a community-editable fluorescent protein database. Nature Methods. 16, 277-278. doi: 10.1038/s41592-019-0352-8).

In a preferred embodiment of the present invention the fluorescent labels are extrinsic fluorescent labels.

Extrinsic fluorescent labels can include but are not limited to commercially available labels, such as Cyanine dyes including Cy5, Cy3, Atto647, Atto647N, Alexa647 Dy647, and the like.

Preferred extrinsic fluorescent labels are environment sensitive dyes described for example in WO 2018/234557, which is incorporated herein by reference. Environment sensitive dyes are known in the art and are described for example in Klymchenko, A. S. (2017) (Solvatochromic and fluorogenic dyes as environment-sensitive probes: design and biological applications. Accounts of chemical research, 50(2), 366-375.). In particular, WO 2018/234557, relates to fluorescent labels which are highly sensitive to environmental changes, e.g., changes in the chemical composition, temperature changes and the like. According to a preferred embodiment, these dyes are selected from the group consisting of the NanoTemper RED, GREEN, and BLUE dyes (commercially available e.g., as Protein Labeling Kits from NanoTemper Technologies GmbH, Munich, Germany).

By labeling with an extrinsic fluorescent label, it can be controlled that only one dye is attached to a target molecule and only that dye needs to be affected by ligand binding. Furthermore, the labeling chemistry can be tailored so that the dye can be placed in a location that is optimal for detecting changes in the chemical environment (e.g., in the proximity of the ligand binding site). Moreover, extrinsic dyes are usually located on the protein surface and therefore have an ideal exposure to sense changes to the chemical microenvironment. The dye fluorescence range can be chosen so that it does not interfere with auto-fluorescence of a ligand. Lastly, extrinsic dyes are much brighter, and measurements can occur at much lower concentration of the target molecule, thus reducing sample consumption and allowing the measurement of even picomolar affinities.

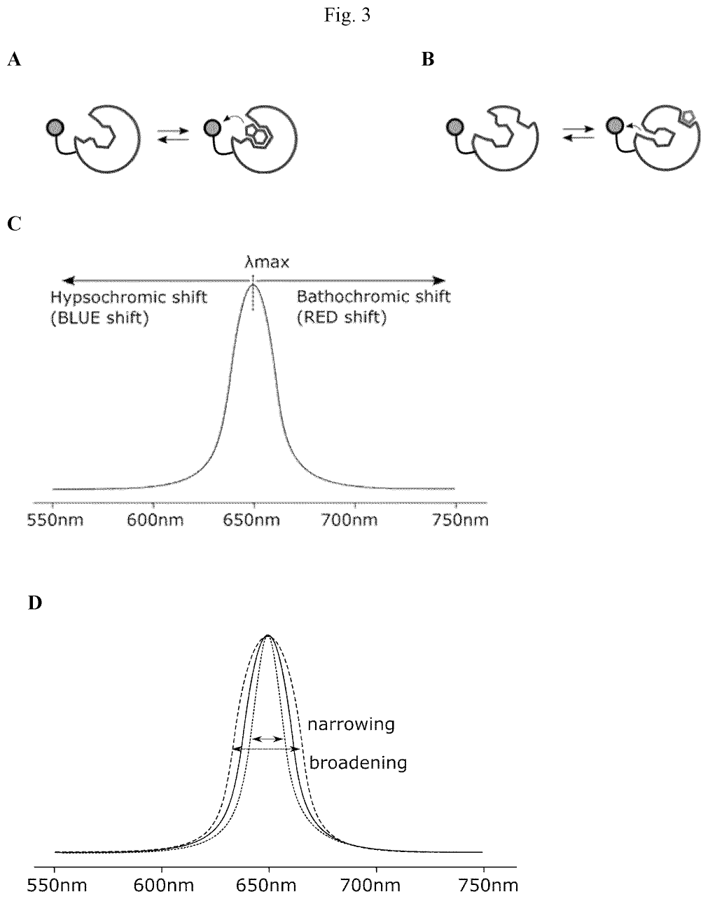

Alterations in the fluorescence spectrum according to the present invention include changes in the fluorescence intensity of a fluorescent label, but also include spectral shifts (see FIG. 3C) and/or broadening or narrowing of their spectrum (see FIG. 3D). According to the present invention, it is preferred to detect the fluorescence at different wavelengths or wavelengths ranges, which could be achieved, e.g., by using bandpass filters. The detected intensities at these different wavelengths/wavelength ranges and the respective ratio allow a detection of a spectral shift of the entire emission spectrum, a broadening and/or narrowing of the spectrum.

In the context of the present invention the spectral shifts preferably include bathochromic (i.e. red) shifts and/or hypsochromic (i.e. blue) shifts.

In the context of the present invention the magnitude of the spectral shifts is preferably at least 50 pm, more preferably at least 100 pm, even more preferably at least 500 pm.

According to the present invention, the fluorescence intensity of the fluorescently labeled particles preferably changes due to mechanisms selected from the group consisting of conformational changes of the fluorescently labeled particles, re-localization of the fluorescently labeled particles, interactions between the fluorescently labeled particles and one or more ligands or combinations thereof and the like.

### Sample Chambers

The sample to be used in the present invention is preferably provided in a sample chamber preferably selected from the group consisting of capillaries, multi-well plates, a microfluidic chip, a cuvette, a reaction tube, a pipette tip, microfluidics, droplets, native tissues, organelles, 3D printed tissues, 3D printed organelles, and a translucent container. The translucent container can be a glass container or a plastic container.

In a preferred embodiment of the present invention the sample containing the fluorescently labeled particles is provided in a capillary.

In another preferred embodiment of the present invention the sample containing the fluorescently labeled particles is provided in a multi-well plate, for example a 96 well, a 384 well or a 1536 well plate.

Preferably, the capillary is made of glass and/or a polymer and/or at least one of the elements of borosilicate glass, borosilicate 3.3 glass (for example DURAN-glass), quartz glass like suprasil, infrasil, synthetic fused silica, soda-lime glass, Bk-7, ASTM Type 1 Class A glass, ASTM Type 1 Class B glass. The polymers may comprise PTFE, PMMA, Zeonor™ Zeonex™, Teflon AF, PC, PE, PET, PPS, PVDF, PFA, FEP, and/or acrylic glass.

In particular, it is preferred that at least one range of the capillaries is transparent for light having a wavelength of 200 nm to 1000 nm, preferably from 250 nm to 900 nm. Particularly preferred, but not limited thereto, said range of the capillary is also transparent for light having the following wavelength ranges: from 940 nm to 1040 nm (preferably 980 nm+/−10 nm), from 1150 nm to 1210 nm, from 1280 nm to 1600 nm (preferably 1450 nm+/−20 nm and/or 1480 nm+/−20 nm and/or 1550 nm+/−20 nm), from 1900 nm to 2000 nm (preferably 1930 nm+/−20 nm). The skilled person understands that the transparent range(s) may also extend over the complete capillary. In other words, the capillaries may be transparent and are preferably made integrally of one of the above-mentioned materials.

Preferably, the used capillaries have an inner diameter of 0.1 mm to 0.8 mm, preferably 0.2 mm to 0.6 mm, further preferably 0.5 mm. The outer diameter of preferred capillaries is preferably between 0.2 mm to 1.0 mm, preferably 0.3 mm to 0.65 mm.

The geometry of the capillaries is not limited to a certain shape. Preferably, tube-like capillaries having a round cross-section or an oval cross-section are used. However, it is also possible to use capillaries having a different cross-section, for example, triangular, quadrangular, pentagonal or polygonal. Preferably, a capillary comprises one of the specific cross sections over the entire length of the capillary. Moreover, it is further preferred that the inner and/or outer dimension of the capillary is constant along the entire length of the capillary. For instance, it is preferred that a cylindric (tubular) capillary comprises the same inner and same outer diameter along the entire length of the capillary. In other words, capillaries may be used which have a diameter and/or cross-section which is constant or not constant over the length of the capillaries.

Particularly, the sample chambers used for the present invention exhibit low autofluorescence, over a broad spectral range. Said autofluorescence is preferably lower than 20%, more preferably lower than 10%, even more preferably lower than 5%.

It is advantageous to provide the sample probe within a chamber which has a thickness in direction of the fluorescence excitation beam from 1 μm to 20 mm, in particular from 1 μm to 6 mm, in particular 1 μm to 500 μm, in particular 1 μm to 250 μm, in particular 1 μm to 100 μm, in particular 3 μm to 50 μm, in particular 5 μm to 30 μm. A person skilled in the art will understand that the term chamber also relates to e.g., a capillary, microfluidic chip or multi-well plate.

### Silicon Surface

Preferred surfaces on which the sample chambers are placed are described for example in WO 2017/055583, which is herein incorporated by reference. In particular, WO 2017/055583 relates to a silicon surface on/above which the sample chambers (e.g., capillaries) of the present invention are preferably placed.

### Sample Volume

Typically, the sample volume containing the fluorescently labeled particles is less than 500 μl, preferably less than 200 μl, more preferably less than 100 μl, even more preferably between 1 μl and 25 μl.

### Sample

Typically, the sample to be used in the method of the present invention is a solution comprising fluorescently labeled particles and ligands. Herein, the labeled particles can be dissolved or dispersed in the solution.

The labeled particles can be immobilized on a solid support, which is brought into contact with the solution containing the ligands. Preferably, the labeled particles are dissolved or dispersed in the solution selected from the group consisting of organic solutions and/or aqueous solutions, particularly buffered aqueous solutions. The buffered aqueous solution is preferably adjusted to a pH value of 2 to 10, more preferably 4 to 10, even more preferably 5 to 9, most preferably 6 to 8.5, using a buffer.

### Fluorescence Measurement (Excitation/Emission)

According to the present invention, preferable means for exciting, preferably fluorescently exciting the labeled particles/molecules may be any suitable device selected from the group consisting of laser, fibre laser, diode-laser, light emitting diodes (LEDs), Halogen, LED-Array, HBO (HBO lamps are, e.g., short arc lamps in which the discharge arc fires in an atmosphere of mercury vapour under high pressure), HXP (MCP lamps are, e.g., short arc lamps in which the discharge arc burns in an atmosphere of mercury vapour at very high pressure e.g., in contrast to HBO lamps they are operated at a substantially higher pressure and they employ halogen cycle. HXP lamps generate UV and visible light, including significant portion of red light) and the like.

Preferably, in the context of the present invention the excitation light source enables a highly focused excitation. In the context of the present invention the excitation light source is preferably a laser, even more preferably an LED.

It is understood by the person skilled in the art that the term “fluorescence” as employed herein is not limited to “fluorescence” per se but that the herein disclosed means, methods and devices may also be used and employed by usage of other means, in particular luminescence, such as phosphorescence. Accordingly, the term of step b) “exciting the fluorescently labeled particles at a first wavelength” relates to the “excitation step” in the above identified method and may comprise the corresponding excitation of luminescence, e.g., excitation is carried out at a shorter wavelength than the detection of the following emission. Therefore, the term “detecting the fluorescence emission intensities of the fluorescently labeled particles at second and a third wavelength” in context of this invention means a step of detection said emissions after excitation. The person skilled in the art is aware that in the context of this invention the “excitation” wavelength and the “emission” wavelengths have to be separated. Additionally, the person skilled in the art is aware that in the context of the present invention, the detected third wavelength is different to the detected second wavelength. The two signals needed for the ratiometric analysis can be obtained by either using a “dual-excitation” configuration or a “dual-emission” configuration.

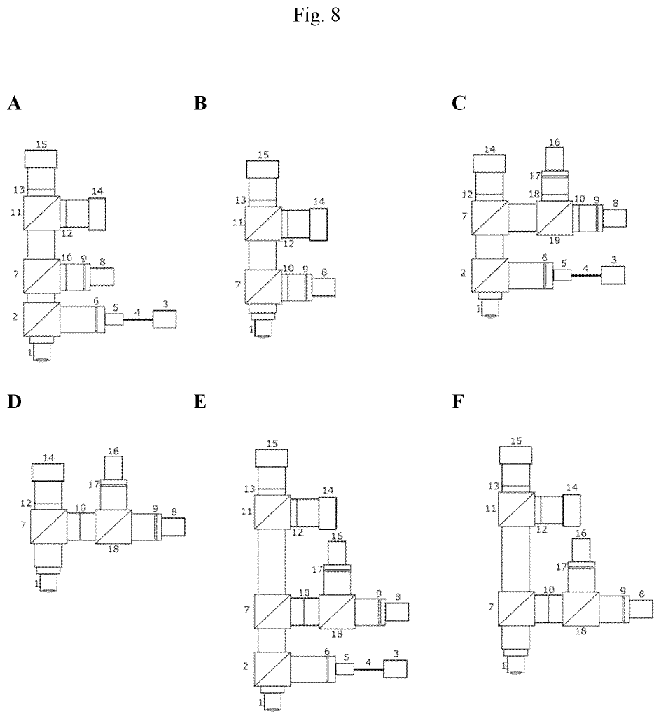

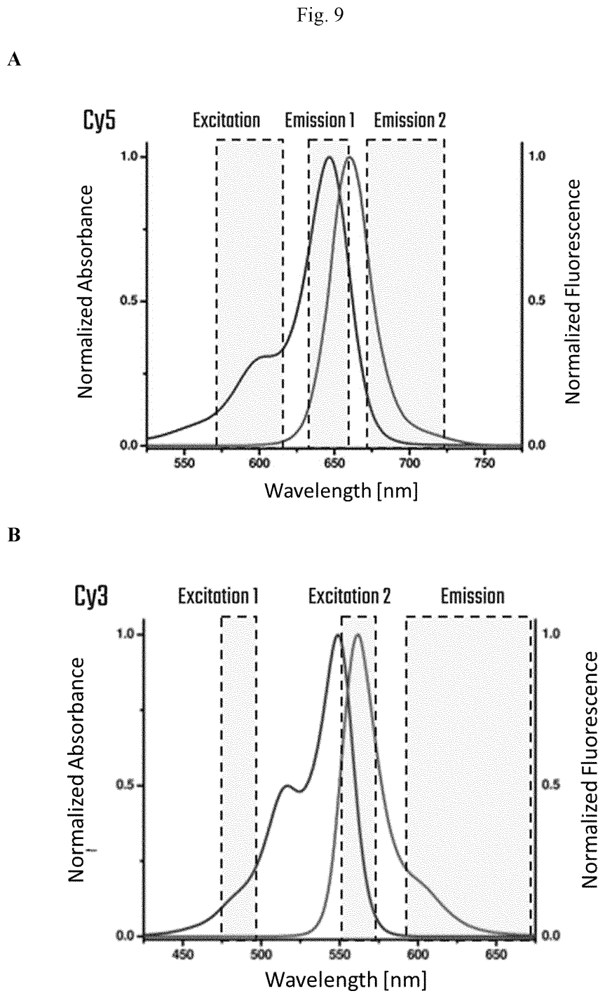

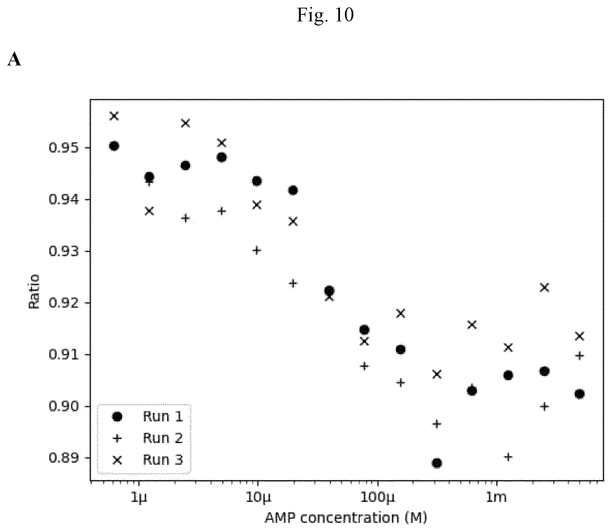

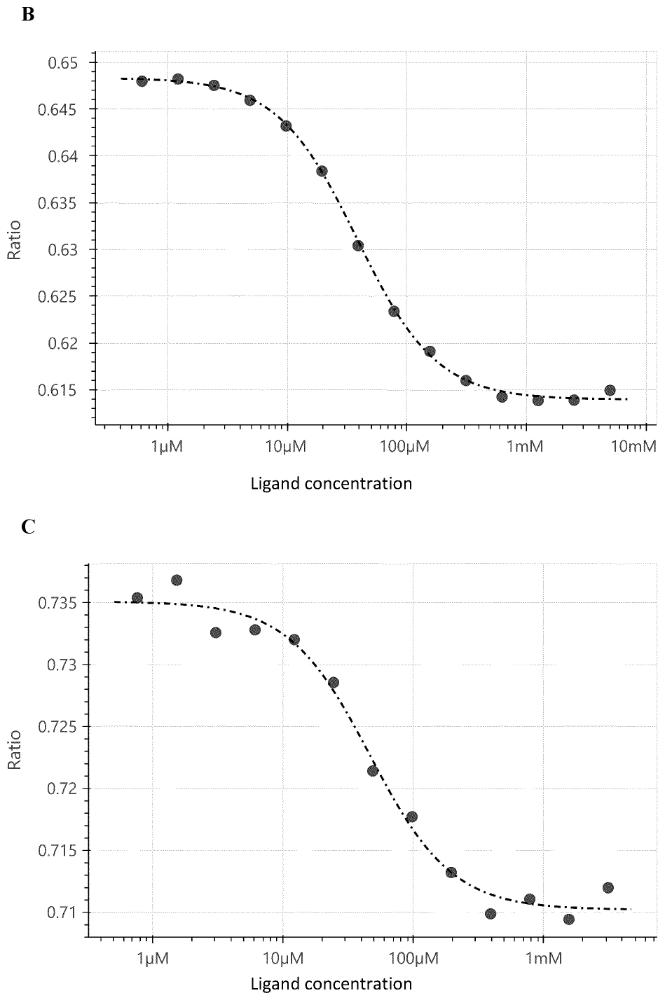

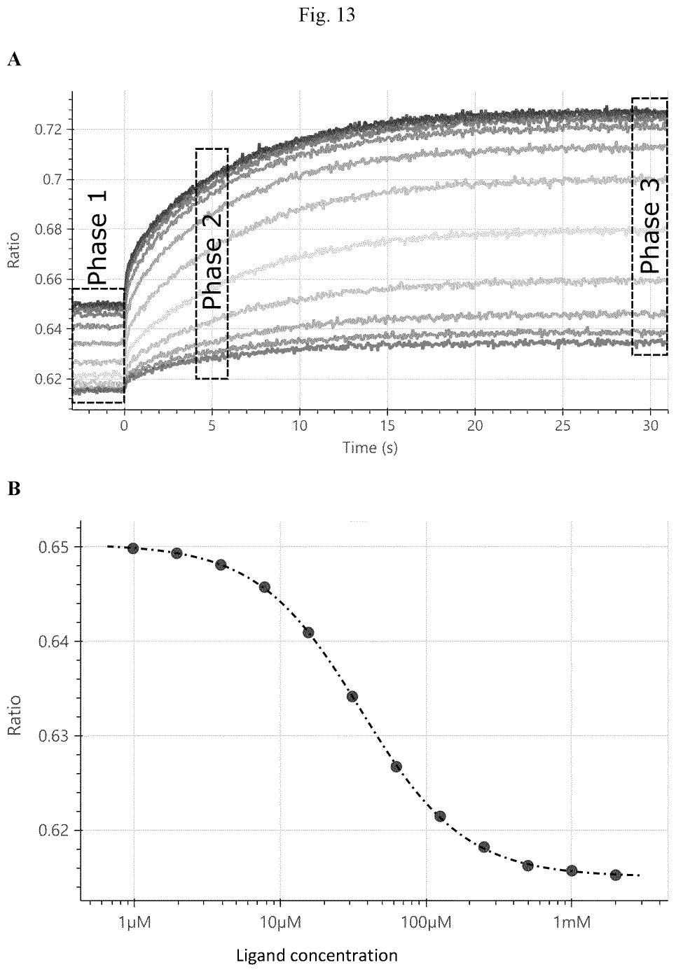

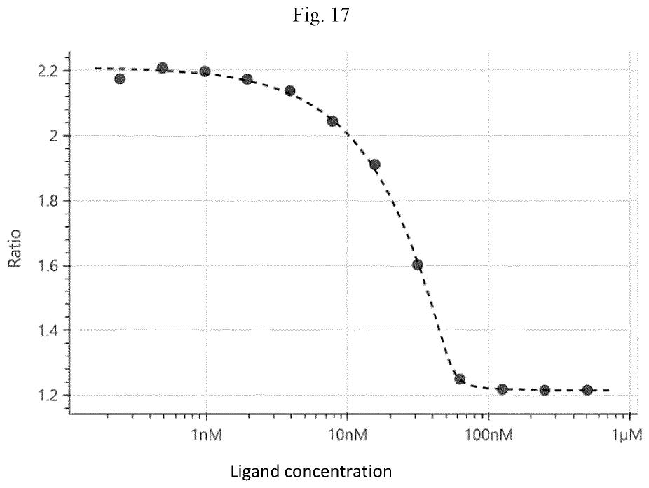

Typically, the ratiometric analysis is based on the “dual-emission” configuration e.g. by using the exemplary dual-emission optics provided in FIG. 8B. Particularly, the one or more samples containing the fluorescently labeled particles are excited at a single constant wavelength, and their emission spectrum is detected at two different wavelengths (see Example 1 and 2 in combination with FIGS. 10 and 13, respectively as well as Examples 4 to 12 in combination with FIGS. 17 to 28). By using the “dual-emission” configuration, the two emission signals can be detected at the same position and at the same time. Furthermore, a lot of fluorescent labels (e.g. Cy5) have a smaller second excitation peak, which can be used for efficient excitation while allowing enough bandwidth at greater wavelengths in order to split the emission spectra into two (FIG. 9A).

In a preferred embodiment of the present invention, the “dual-emission” configuration is used in combination with “red” fluorescent labels, such as Cy5, RFP, and the like. Since such labels have their excitation maximum at approx. 650 nm with a secondary excitation peak at approx. 600 nm and the emission maximum at approx. 660 nm, well-suited excitation and emission wavelengths include excitation between approx. 570 nm and 615 nm, detection of the first emission between approx. 625 nm and 650 nm and detection of the second emission between approx. 670 nm and 725 nm (FIG. 9A). Exemplary components, which can be used for the “dual-emission” configuration, are provided in Table 1.

TABLE 1

| | Example | | | | | |
|---|---|---|---|---|---|---|
| | | Manu- | | | Wavelength | |
| Component | Type | facturer | Name | Part Number | Range | |
| Excitation | LED | Cree | LM1-UYL1-11-N1- | LM1-UYL1-11-N1- | 591 | nm |
| Light Source | | | 00001 | 00001 | | |
| Excitation | Band | Semrock | 593/46 BrightLine HC | F39-593 (FF01- | 570 . . . 615 | nm |
| filter | pass | | AOI 0°, Diameter 25 | 593/46-25) | | |
| | | | mm, Ring thickness | | | |
| | | | 3.5 mm | | | |
| Light | Dichroic | Custom | Beam splitter T | F48-622 | 615 . . . 626 | nm |
| separation | mirror | by AHF | 622LPXR | (T622LPXR) | | |
| element | | | AOI 45°, Reflexion | | | |
| | | | 465-615 nm >90%, | | | |
| | | | Transmission 626-900 | | | |
| | | | nm >90%, dimensions | | | |
| | | | 25.5 × 36 × 1 mm | | | |
| Second light | Dichroic | Semrock | Beam splitter T | F48-660 | 653 . . . 661 | nm |
| separation | mirror | | 660LPXR | (T660LPXR) | | |
| element | | | AOI 45°, Reflexion | | | |
| | | | 500-653 nm >90%, | | | |
| | | | Transmission 661-800 | | | |
| | | | nm >90%, dimensions | | | |
| | | | 25.5 × 36 × 1 mm | | | |
| Emission | Band | Semrock | 640/20 BrightLine HC | F39-641 (FF01- | 628 . . . 653 | nm |
| filter | pass | | AOI 0°, Diameter 25 | 640/20-25) | | |
| | | | mm, Ring thickness | | | |
| | | | 3.5 mm | | | |
| Second | Band | Semrock | 697/58 BrightLine HC | F37-697 (FF01- | 665 . . . 727 | nm |
| emission | pass | | AOI 0°, Diameter 25 | 697/58-25) | | |
| filter | | | mm, Substrate | | | |
| | | | thickness 1.05 mm, | | | |
| | | | Ring thickness 3.5 mm | | | |
| Detectors | PMT | Hamamatsu | H10722-20 | H10722-20 | 230 . . . 920 | nm |

According to the present invention, the second wavelength is preferably detected at a shorter wavelength and the third wavelength is detected at a longer wavelength than an emission maximum of the fluorescently labeled particles under the first condition. The person skilled in the art is aware that in the context of the present invention an emission maximum can be a local emission maximum or an absolute emission maximum. Alternatively, the detection can be around a saddle point of the emission spectrum instead of an emission maximum.

Small changes (e.g., wavelength shifts) in the regions flanking the emission maximum have a relatively large impact on the alterations of a fluorescence spectrum (e.g., in terms of intensities).

Thus, in a preferred embodiment of the present invention the emission fluorescence intensities are detected in close proximity to an emission maximum, e.g., the second wavelength is detected at an at least 2.5 nm shorter wavelength (e.g., a 10 nm shorter wavelength) and the third wavelength is detected at an at least 2.5 nm longer wavelength (e.g., 10 nm longer wavelength) than said emission maximum of the fluorescently labeled particles under first conditions.

According to the present invention, preferable means for detecting the excited fluorescently labeled particles, particularly for detecting the fluorescence, may be any suitable device selected from the group consisting of charge coupled device (CCD) cameras (2D or line-scan CCD), Line-Cameras, Photomultiplier Tubes (PMT), silicon photomultipliers (siPMs), avalanche photodiodes (APD), photodiode arrays (PDAs), complementary metal-oxide-semiconductor (CMOS) cameras and the like.

### Alternative Fluorescent Measurement

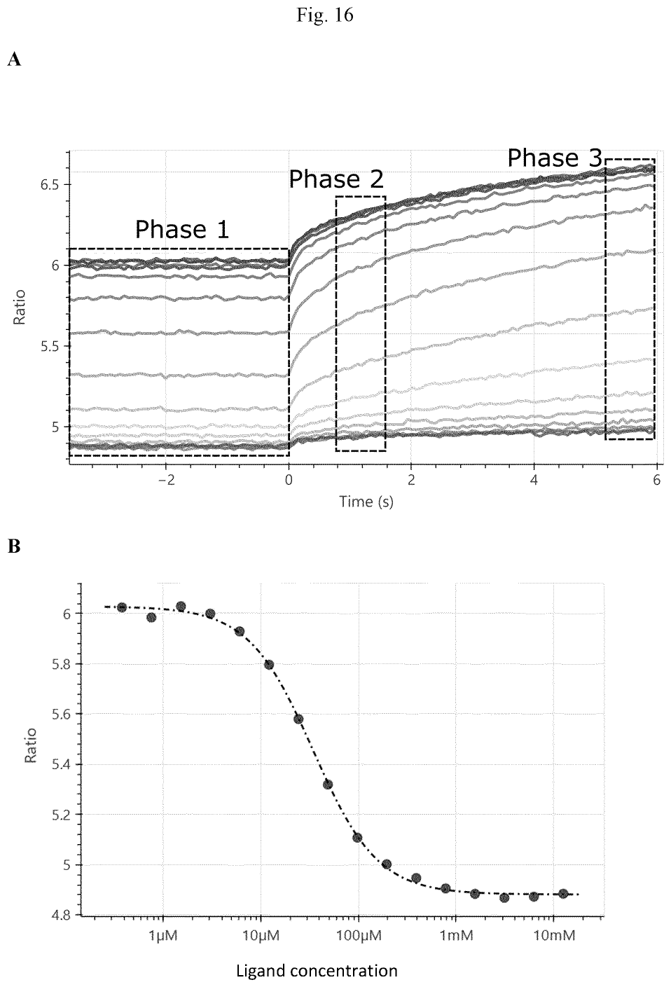

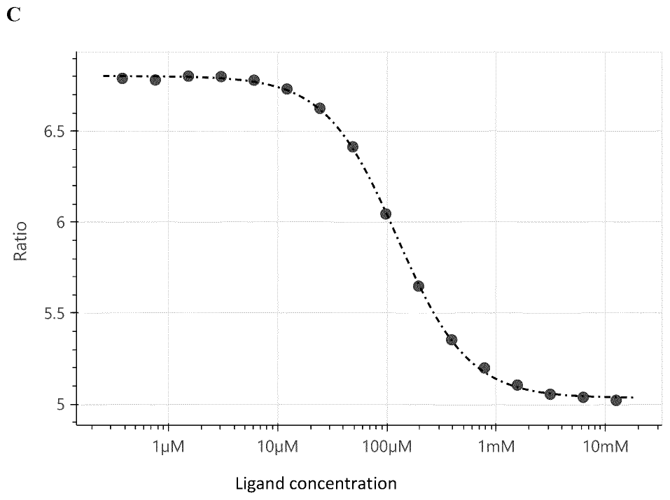

In another embodiment of the present invention, the ratiometric analysis is based on the “dual-excitation” configuration, e.g., by using the exemplary dual-excitation optics provided in FIG. 8D. Particularly, the one or more samples containing the fluorescently labeled particles are excited at two different wavelengths, and their emission spectrum is detected at a single wavelength (see Example 3 in combination with FIG. 16). By using this configuration, the two excitation spectra cannot be detected simultaneously, which means that they need to be acquired subsequently. This approach is more time-consuming and the time-delay between the two subsequent measurements might lead to substantial differences between said measurements, for example, by bleaching events induced after the first excitation, sample aggregation, etc.

By using a “stroboscopic” excitation approach in which the two excitation light sources are switched on and off in less than 1 s or even faster and the data is collected alternatingly, some of the abovementioned problems might be solved. However, the two different excitation light sources can lead to different bleaching rates of the sample, which might have a negative effect on the evaluation of the ratiometric signal when dealing with longer acquisition times.

In another embodiment of the present invention, the “dual-excitation” configuration is used in combination with “green” fluorescent labels, such as Cy3, GFP, and the like. Since such labels have their excitation maximum at approx. 540 nm, and the emission maximum at approx. 560 nm with a secondary emission peak at approx. 600 nm, well-suited excitation and emission wavelengths include excitation between approx. 475 nm and 495 nm, detection of the first emission between approx. 550 nm and 575 nm and detection of the second emission between approx. 590 nm and 680 nm (FIG. 9B). Exemplary components, which can be used for the “dual-emission” configuration, are provided in Table 2.

TABLE 2

| | Example | | | | | |
|---|---|---|---|---|---|---|
| | | Manu- | | | Wavelength | |
| Component | Type | facturer | Name | Part Number | Range | |
| Excitation | LED | Cree | LM1-EBL1-01-N2 | LM1-EBL1-01-N2 | 480 | nm |
| Light Source | | | | | | |
| Second | LED | Cree | LM1-EPG1-01-N2 | LM1-EPG1-01-N2 | 540 | nm |
| Excitation | | | | | | |
| Light Source | | | | | | |
| Excitation | Dual | Chroma | FITC/CY3 ET | F59-023 | 475 . . . 495 | nm |
| filter | band | | Dualband | FITC/CY3 | 550 . . . 575 | nm |
| | | | Excitation filter | | | |
| Second light | Dichroic | Chroma | ET Dualband bs | F58-023 | 500 . . . 540 | nm |
| separation | mirror | | FITC/CY3 | FITC/CY3 | 590 . . . 680 | nm |
| element | | | | | | |
| Emission | Dual | Chroma | FITC/CY3 ET | F57-023 | 590 . . . 680 | nm |
| filter | band | | Dualband Cut filter | FITC/CY3 | | |
| Detectors | PMT | Hamamatsu | H10722-20 | H10722-20 | 230 . . . 920 | nm |

The excitation volume is typically the part of the sample volume which gets fluorescently excited by the excitation light source. The detection volume is the part of the sample volume of which the emission spectrum gets detected. According to the present invention, the excitation volume and/or detection volume is of the size of preferably 2 mm×2 mm×5 mm or less, more preferably of 1 mm×1 mm×5 mm or less, even more preferably 0.5 mm×0.5 mm×5 mm or less.

However, means for exciting the fluorescently labeled particles and detecting the fluorescence of said excited particles are not limited and any suitable means known to the skilled person may be employed.

### Ratiometric Characterization

According to the present invention, the ratiometric analysis is preferably based on building the ratio between the fluorescence intensities detected at the second and third wavelength by pointwise division. The person skilled in the art is aware that in the context of this invention the ratio of said fluorescence intensities can be obtained by dividing either the third wavelength by the second wavelength or vice versa (e.g., the second by the third wavelength).

In the context of the present invention exemplary relative percentual changes of the ratio (i.e., the percentual change of the ratio after a spectral shift) are at least 0.5%, at least 1.1%, at least 5.5%, at least 37.3%. Preferably herein, the relative percentual change is at least 3%.

### Characterization of Interactions

In the context of the present invention, the interactions of fluorescently labeled particles includes, in particular biomolecules with e.g., further (bio)molecules, particles, beads, as well as stability of (bio)molecules, their conformation for folding and unfolding or their chemical environment (such as their location within aqueous solutions, a lipid nanoparticle or a cell). Further interaction characterization equilibrium measurements, binding kinetic measurements and measurements of thermodynamic parameters.

According to the present invention, the calculated ratios are preferably used to determine the localization of the fluorescently labeled particles or parameters selected from the group consisting of dissociation constants, half maximal effective concentrations (EC₅₀), equilibrium constants, binding kinetics, enzymatic reaction kinetics, thermodynamic parameters, stability parameters (e.g. thermal denaturation of proteins, chemical denaturation of proteins and the like), unfolding or refolding kinetics, opening and closing reactions, and/or combinations thereof and the like.

In context of the present invention the skilled person is aware that the dissociation constant (K_{d}) of an interaction can be determined from the obtained fluorescence intensity ratio by fitting the data with the Langmuir equation and the half maximal effective concentrations (EC₅₀) can be determined from the obtained fluorescence intensity ratio by fitting with the Hill equation (Ganellin, C. R., Jefferis, R., & Roberts, S. M. (Eds.). (2013). Introduction to biological and small molecule drug research and development: theory and case studies. Academic Press., chapter 1, page 38 and 39).

According to the present invention, the first and second conditions of the fluorescently labeled particles may differ with regard to their chemical composition and/or temperature and/or localization within a chemical macroenvironment (e.g. the first conditions of the fluorescently labeled particles relates to the location of the particles within a first carrier, e.g. a vector and the second conditions relate to the location of the particles within a second carrier, e.g. a recipient or in a buffer solution containing both carriers).

The fluorescence spectrum of a fluorescent label according to the present invention can also change when the fluorescently labeled particle/molecule is in complex with one or more other molecules, e.g., ligands (e.g., through ligand proximity (see FIG. 3B) and/or conformational changes upon binding of a ligand (see FIG. 3B).

Thus, in a preferred embodiment of the present invention, the second conditions may be altered by adding a ligand and/or different concentrations of the ligand and the calculated ratios obtained are used to determine a dose-response curve and the dissociation constant (K_{d}) of the fluorescently labeled particles and the ligand.

In the context of the present invention, the term “binding” of the ligand to the labeled particle, preferably refers to covalent binding or binding by intermolecular forces such as ionic bonds, hydrogen bonds and van der Waals forces.

Ligand binding to a target biomolecule like a protein can result in a wide range of conformational changes such as amino acid side chain, loop or domain movement. The ligand which can be used according to the present invention can be (but not limited to) selected from the group consisting of ions, metals, compounds, drug fragments (small chemical fragments, which may bind only weakly to the biological target), carbohydrates, small molecules (organic compounds having a low molecular weight (<900 Daltons); small molecules may help regulate a biological process and usually have a size on the order of 1 nm), drugs, prodrugs, lipids, proteins, peptides, peptoids, enzymes, nucleic acids, aptamers, nanoparticles, liposomes, unilamellar vesicles (including small unilamellar vesicles (SUV) and giant unilamellar vesicles (GUV)), polymers, organic molecules, inorganic molecules, metal complexes, hormones, flavors, odorants, particles and (micro)beads. Preferably, the ligands are selected from the group consisting of ions, metals, compounds, drug fragments, carbohydrates, small molecules, drugs, prodrugs, lipids, proteins, peptides, peptoids, enzymes, nucleic acids, aptamers, hormones, flavors, and odorants.

The concentration of the ligand is preferably from 0.01 pM to 1 M, preferably 1 pM to 100 mM, more preferably 1 pM to 10 mM

### Combination with Temperature Related Intensity Change (TRIC) and Micro-Scale Thermophoresis (MST)

Although the ratiometric analysis of fluorescently labeled particles according to the present invention does not necessarily rely on temperature induced fluorescence intensity changes, the fluorescence intensity measurement may be performed at a constant predetermined temperature or during a defined temperature perturbation.

In a second aspect, the present invention relates to a method for the characterization of fluorescently labeled particles in solution by analyzing alterations in the fluorescence spectrum of the fluorescently labeled particles in combination with a defined temperature perturbation.

The method of the second aspect of the invention comprises the following steps:

a) providing a sample of the fluorescently labeled particles in a solution under first conditions,

b) exciting the fluorescently labeled particles at a first wavelength,

c) detecting the fluorescence emission intensity of the fluorescently labeled particles at a second and a third wavelength,
wherein said intensities are detected during a defined temperature perturbation.

d) calculating the ratio between said fluorescence intensities at the second and the third wavelength, wherein said third wavelength is different from said second wavelength,

e1) repeating steps b) to d) for said sample of the fluorescently labeled particles under second conditions, or

e2) repeating the steps a) to d) for a second sample of the fluorescently labeled particles under second conditions,

wherein said second conditions are different from the said first conditions,

f) characterizing the fluorescently labeled particles based on the calculated ratios obtained for the different conditions,

wherein the second and third wavelength are detected simultaneously, and wherein the second wavelength is shorter and the third wavelength is longer than an emission maximum of the fluorescence emission of the fluorescently labeled particles under the first conditions.

In the method of the preferred embodiment of the second aspect of the present invention, the heating or cooling can be carried out using a tempering element (i.e. a heating and/or cooling source) selected from the group consisting of heating and/or cooling fluids or gases, heating elements (for example a heating resistor, or other elements based on joule heating like Metal heating elements, Ceramic heating elements, Polymer PTC heating elements, Composite heating elements, semiconductor heating elements), or a thermoelectric element, for example a Peltier element, or electromagnetic radiation (like an LED, e.g., an IR-LED, or a laser, e.g., an IR laser, or a microwave). The use of an IR laser enables fast sample heating.

A Peltier element is preferably used because it can be used to heat the sample and/or to cool the sample (e.g., to cool the sample below the environmental temperature). In particular, it is possible to switch from heating to cooling by reversing the direction of the current through the Peltier element. A Peltier element is one of the few elements that can heat but also actively cool under room temperature.

A laser, preferably a laser whose electromagnetic radiation is directly absorbed by the sample, is preferably used because the temperature can be changed rapidly and directly in the sample without mechanical contact to the sample.

Also preferred is that said laser is a high-power laser within the range of from 0.01 W to 10 W, preferably from 4 W to 6 W.

Also preferred is that said laser is a laser within the range of from 1 mW to 1 W, preferably from 1 mW to 500 mW, more preferably from 1 mW to 250 mW.

Laser radiation is directly absorbed by the sample and converted to heat, e.g., IR laser light of the wavelengths 980 nm+/−30 nm, 1480 nm+/−30 nm, 1550 nm+/−30 nm, 1940 nm+/−30 nm is very well absorbed by water and heats up very quickly. This heating method is contact-less and may thus be fast and without the risk of contamination. The sample chamber must only be transparent to the laser light but does not require a good thermal conductivity, in contrast to contact heating by means of a heating element.

With an IR laser, very small volumes (e.g. in the nanoliter volume range) can be heated up, of which the fluorescence is measured by fluorescence optics (typically only 100 μm×100 μm×100 μm=1 nl volume).

According to the present invention the samples to be investigated may also be subjected to linear temperature ramps by heating and/or cooling the tempering element at defined constant rates e.g. 1° C./min or 1 K/min. Typically the heating and/or cooling rates are between 0.1 K/min to 50K/min using contact heating for example with Peltier elements.

In another embodiment the samples can be heated with an IR laser (“optical heating”) with typical heating rates of 1 K/s to 100 K/s.

The method according to the second aspect of the present invention is preferably performed within a temperature range of −20° C. to 160° C., more preferably of 0° C. to 120° C.

The preferred data acquisition time for the measurement of the initial ratio is between 1 s and 5 s, the preferred data acquisition time for the ratio obtained during temperature perturbation is between 5 s and 20 s, but the acquisition times can also be shorter, for example only 10 ms to 100 ms, or longer, for example minutes, hours or even days.

The ratio can also be analyzed at any later time of the temperature change. This may be beneficial when the amplitude is very small at room temperature but increases at higher temperatures (see Example 2 in combination with FIG. 13A, in which the analysis at phase 3 results in a larger amplitude than the analysis at phase 1).

In a preferred embodiment of the second aspect of the present invention, the ratiometric analysis is based on the “dual-emission” configuration in combination with a temperature perturbation, i.e., by using the exemplary dual-emission optics and an IR laser provided in FIG. 8A.

In another embodiment of the present invention, the ratiometric analysis is based on the “dual-excitation” configuration in combination with a temperature perturbation, e.g., by using the exemplary dual-excitation optics and an IR laser provided in FIG. 8C.

In another embodiment of the present invention, the ratiometric analysis is based on both the “dual-excitation” and the “dual-emission” configuration, i.e., by using the exemplary “dual-excitation/dual-emission” optics provided in FIG. 8F.

In another embodiment of the present invention, the ratiometric analysis is based on both the “dual-excitation” and the “dual-emission” configuration in combination with a temperature perturbation, e.g., by using the exemplary “dual-excitation/dual-emission” optics and an IR laser provided in FIG. 8E.

### Determination of Thermodynamic and Kinetic Parameters by Combining the Ratiometric Method with TRIC/MST

In a third aspect, the present invention relates to a method for the characterization of the thermodynamic and/or kinetic parameters of fluorescently labeled particles in solution by analyzing alterations in the fluorescence spectrum of the fluorescently labeled particles in combination with a defined temperature perturbation/temperature change.

In the context of the present invention, thermodynamic parameters include but are not limited to enthalpy, entropy, heat capacity (c_{P})

In the context of the present invention, kinetic parameters include but are not limited to equilibrium constants, dissociation rates, association rates, enzymatic rates, folding and unfolding rates, release rates (e.g., payload release rates in case of LNPs), rate of aggregation, intruding rate (e.g., rate with which a payload like mRNA intrudes a cell).

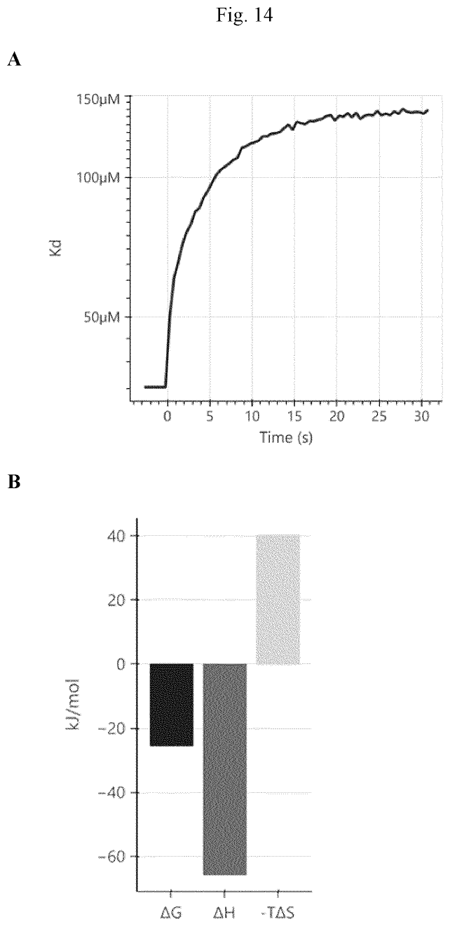

According to the third aspect of the present invention, preferably thermodynamic parameters of interactions can be determined when collecting the ratiometric fluorescence data with an IR laser from a single measurement. Since the sample temperature at each timepoint of the measurement is known (determination in a calibration measurement, in which the tray is heated in a controlled way and the fluorescence of a reference label is measured), a dissociation constant K_{d} can be obtained for each timepoint (see Example 2 in combination with FIG. 14A).

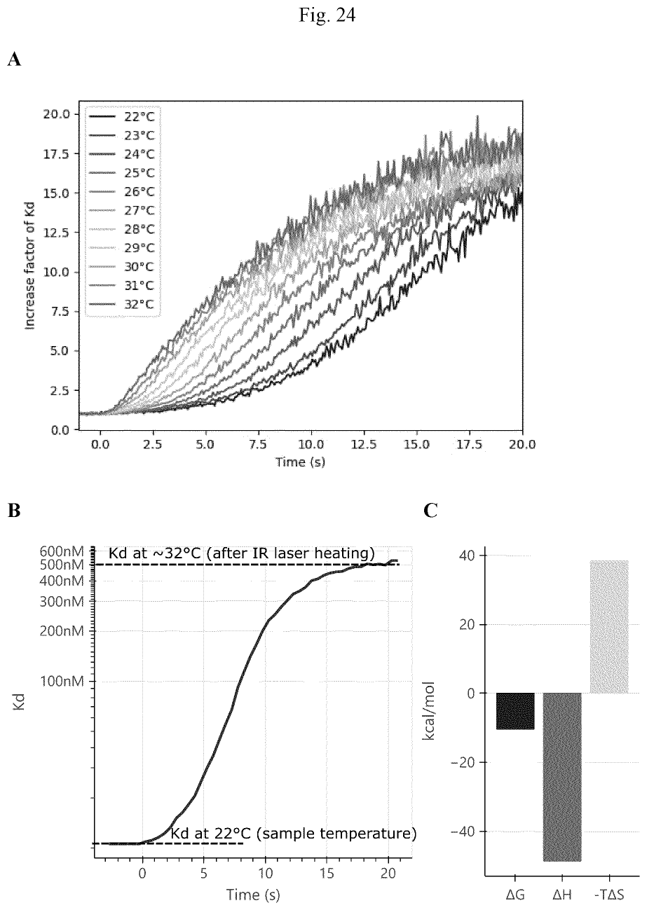

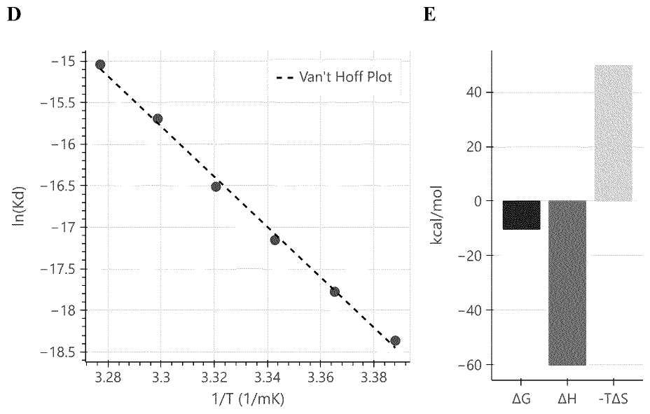

It is added, that according to the third aspect of the present invention, the thermodynamics of a rection can also be determined by performing classical K_{d} measurements at different, fully equilibrated sample temperatures, for example by subsequently setting the sample temperature to 22° C., 24° C., 26° C., 28° C., 30° C., and 32° C. and performing a binding affinity measurement for each temperature (see Example 10 in combination with FIG. 24).

Even if the accuracy of this approach does not always reach the accuracy of a state-of-the-art isothermal titration calorimetry (ITC) measurement, the present measurement is approx. 100× faster and the much lower sample consumption may outweigh the lack of accuracy. For example, for certain molecules the information if being an entropic or enthalpic binder can be highly valuable especially during early developmental stages.

According to the third aspect of the present invention, preferably thermodynamic parameters of interactions can be determined when collecting the ratiometric fluorescence data with an IR laser from a single measurement.

### Application: Monitoring the Localization of Fluorescently Labeled mRNA by Combining the Ratiometric Method with TRIC/MST

In a fourth aspect, the present invention preferably relates to a method for the characterization of the localization of fluorescently labeled particles in solution by analyzing alterations in the fluorescence of the fluorescently labeled particles in combination with or without a defined temperature perturbation/temperature change.

In the context of the present invention, the terms “location” and “localization” are used interchangeable and refer to the determination of the locus/position of fluorescently labeled particles.

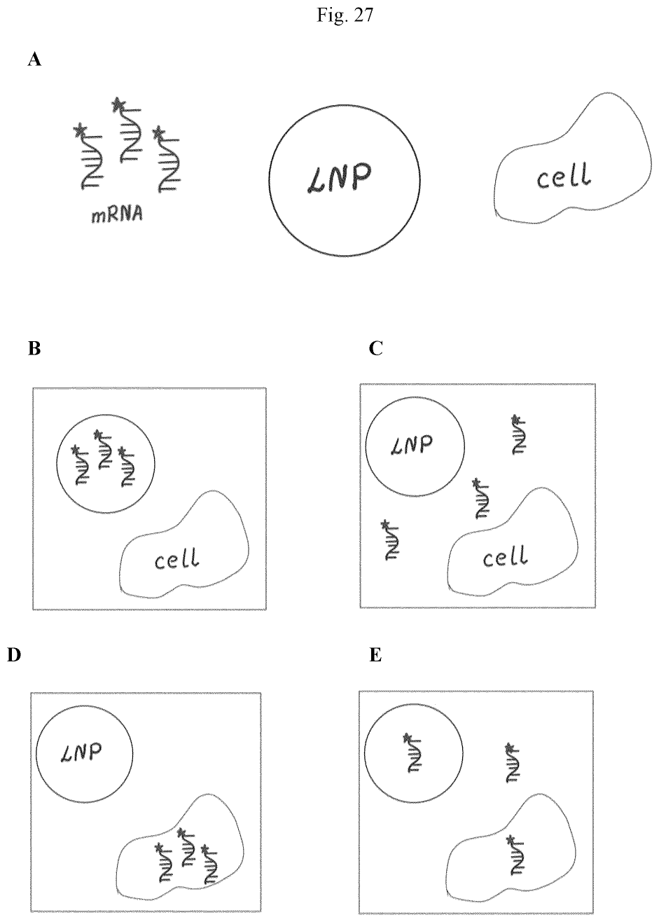

All gene and cell therapy methods involving nucleic acids such as DNA and RNA and the like, have the inherent problem of determining the success of drug delivery (e.g., the delivery of the carried nucleic acid into the target cell via incorporation and release from carriers such as lipid nanoparticles (LNPs) dissolved in a buffer solution; see FIG. 27A).

Even before determining the success of delivery, the bioproduction (e.g., the loading of delivery systems including unloaded, partially loaded, fully loaded and/or overloaded state of LNPs is a crucial step, which needs to be thoroughly evaluated.

According to the fourth aspect of the present invention, carriers can be selected from the group consisting of metal nanoparticles and nanoconstructs, polymeric nanoparticles, lipid-based carriers systems (e.g. liposomes, other lipid-containing complexes), carbonaceous carriers, nanoemulsions, nanosuspensions, nanomicelles, dendrimers, milk-derived carriers, endosomes, viral vectors (e.g. adenoviruses, adeno-associated viruses (AAV), retroviruses), virus like particles (VLPs), eukaryotic cells, prokaryotic cells, cellular fragments, and the like. In view of the above, combinations of said carriers are also within the scope of the present invention.

In a preferred embodiment of the fourth aspect of the present invention, the fluorescently labeled particle is a fluorescently labeled mRNA and the carrier is an LNP.

An LNP refers to any particle having a diameter of less than 1000 nm, 500 nm, 250 nm, 200 nm, 150 nm, 100 nm, 75 nm, 50 nm, or 25 nm. For example, in an appropriate buffer an LNP filled with mRNA can have a hydrodynamic diameter between 65 nm and 85 nm. Alternatively, a nanoparticle may range in size from 1-1000 nm, 1-500 nm, 1-250 nm, 25-200 nm, 25-100 nm, 35-85 nm, or 25-60 nm.

LNPs may be made from cationic, anionic, or neutral lipids. Neutral lipids, such as the fusogenic phospholipid DOPE or the membrane component cholesterol, may be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs may also be comprised of hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1.

Examples of neutral lipids are: DPSC, DPPC, POPC, DOPE, and SM. Examples of PEG-modified lipids are PEG-DMG, PEG-CerC14, and PEG-CerC20.

Since the fluorescence spectrum of a fluorescent label is highly sensitive to environmental changes, such as changes in the chemical environment, and said spectrum alters upon environmental changes, the location of fluorescently labeled particles (e.g., mRNA) can be inferred from the ratiometric measurement according to the present invention.

As an example for a not limiting combination of dyes and a second and a third emission wavelength: If all fluorescently labeled mRNA molecules are properly located within LNPs the ratio after characterization according to the present method corresponds to 2.1 (see Example 12 in combination with FIG. 27B). If all fluorescently labeled mRNA molecules are located outside the LNPs the obtained ratio corresponds to 1.2 (see Example 12 in combination with FIG. 27C).

Since the chemical environment within a cell is very different to the one in a LNP or a buffer solution, a person skilled in the art is aware that in the context of the present invention, the obtained ratio is different to above mentioned values (i.e., 1.2 and 2.1) when all fluorescently labeled mRNA molecules are successfully delivered into the target cell (see Example 12 in combination with FIG. 27D). Furthermore, in case of intermediate states, e.g., fluorescently labeled mRNA molecules are partially located in the cell, the LNP and the buffer solution, the obtained fluorescence ratio is a linear combination of the ratios obtained for the three abovementioned states.

According to this exemplary aspect of the present invention, the determination of the localization of fluorescently labeled particles (e.g., mRNA) is based on very small sample volumes as well as detection volumes and short experimental procedures. In the same context, presently applied methods known in the art (such as field-flow fractionation and liquid chromatography) are based on different methods and require not only more time, but also much higher sample volumes of, not uncommonly, limited and expensive samples.

### Kits for Biotinylated Molecules

In a fifth aspect, the present invention relates to a kit and the use of a kit for the characterization of fluorescently labeled particles, e.g., particles that are labeled with one or more biotin molecules (i.e., biotinylated particles) in solution according to the methods of the present invention.

The kit includes as main components a defined stochiometric ratio of (i) a (preferably tetrameric) biotin-binding protein which comprises at least two (preferably four) binding sites for biotin and (ii) a linking moiety (herein also designated as “linker”). The kit preferably further comprises an instruction manual explaining the use of the kit in at least one of the methods of the present invention.

According to the fifth aspect of the present invention the biotin-binding protein can be selected from the group consisting of streptavidin, avidin, and mutants thereof. In the context of the present invention mutants include neutravidin, flavidin, divalent streptavidin and the like.

The kit useful in the present invention may include a single vial comprising (i) the biotin-binding protein and (ii) a linker that is preferably modified/labeled with a biotin molecule at one end a fluorescent label at the other end. It is a preferred embodiment of the fifth aspect of the present invention that the biotin-binding protein is preferably a tetrameric protein, even more preferably tetrameric streptavidin.

Streptavidin is a homo-tetramer that has an extraordinarily high affinity for biotin (also known as vitamin B7). It is used extensively in molecular biology and bio-nanotechnology due to the resistance of the streptavidin-biotin complex to organic solvents, denaturants (e.g., guanidinium chloride), detergents (e.g., SDS, Triton), proteolytic enzymes, and extremes of temperature and pH.

It is a further preferred embodiment of the fifth aspect of the present invention that the fluorescent label is attached to a linker, e.g., as described in the art (NPL6).

According to the fifth aspect of the present invention a linker is a polymer, preferably a nucleic acid, preferably a single-stranded nucleic acid, more preferably a single-stranded DNA, even more preferably a single-stranded DNA oligomer (i.e., an oligonucleotide), preferably having a length of 6 to 24 nucleotides. For example, a 12-mer oligo-dT strand can be used.

However, the linker used in the context of the present invention is not limited by length (as long as the linker is long enough so that the fluorophore can reach to the target) and/or type of linker (as long as the type of linker has the property that its length can be adjusted, e.g., DNA, aromatic rings including aryl groups, and the like) and any suitable linker known to the skilled person may be employed to indirectly attach a fluorescent label to the particle to be characterized by the method of the present invention.

A linker for use according to the present invention contains a biotin molecule at one end and a fluorescent label at the other end. In a preferred embodiment of the fifth aspect of the present invention, the linker is modified with biotin at its 3′ end and a fluorescent label at its 5′ end, or vice versa.

Each kit may contain material sufficient for multiple labeling reactions. Depending on the size of the kit and the amount of biomolecule used, enough material for approximately 500 up to 3840 single-point ratiometric characterization experiments can be provided.

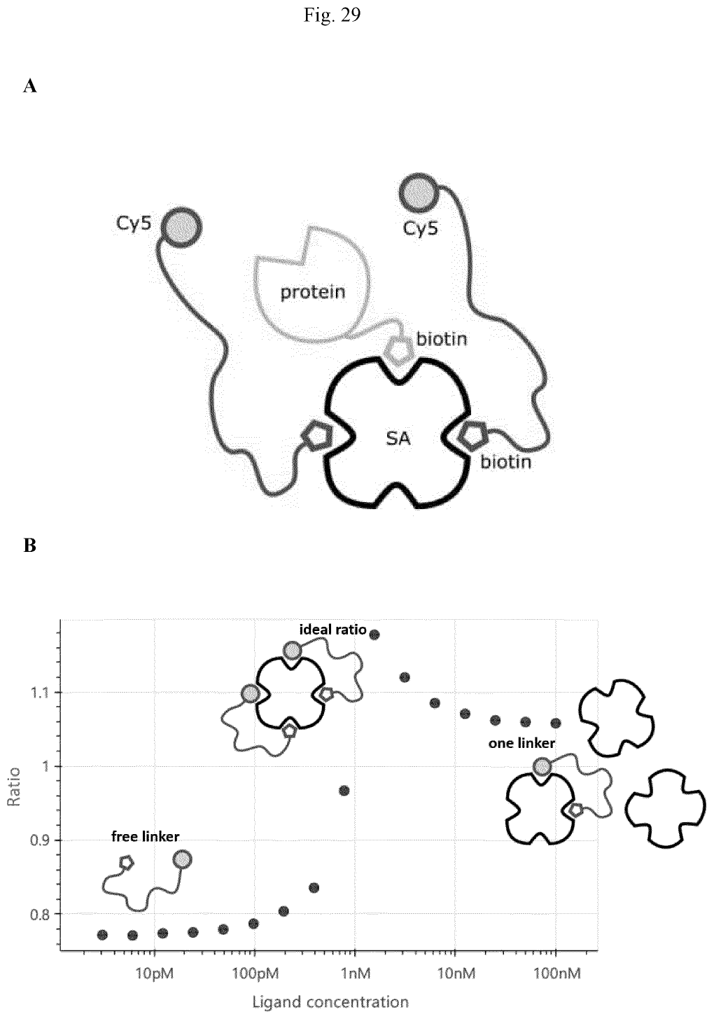

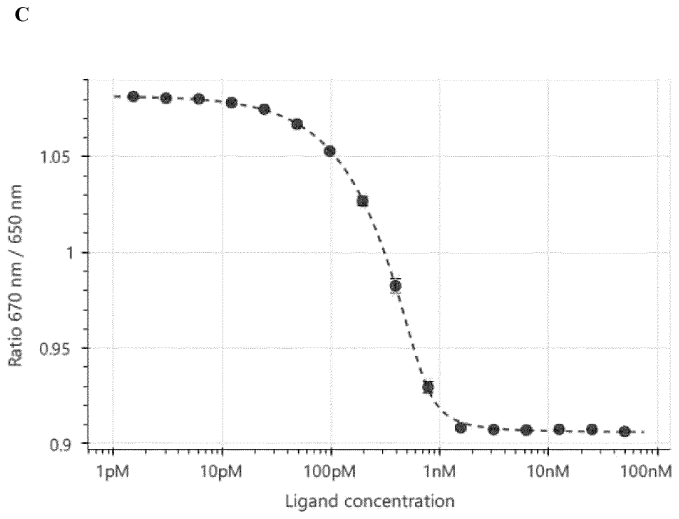

In a preferred embodiment of the fifth aspect of the present invention, the fluorescently labeled particle is a complex between tetrameric streptavidin molecules, biotinylated fluorescently labeled linker molecules and biotinylated target molecules (see FIG. 29A). It is particularly preferred that the fluorescently labeled particle is a complex between one tetrameric streptavidin molecule, two biotinylated fluorescently labeled linker molecules and one biotinylated target molecule.

The stochiometric ratio of tetrameric biotin-binding protein and modified linker is preferably adjusted in a way that that in average two of the four binding sites on the tetrameric biotin-binding protein are occupied (e.g., by supplying the vial with 2 nM streptavidin and 4 nM linker) and the remaining two binding sites are available for binding events with biotinylated particles of interest.

Thus, it is further preferred in the fifth aspect of the present invention that the tetrameric streptavidin and the linker are mixed in a 1:2 ratio, in a way that in average one streptavidin molecule is labeled with two linker molecules, i.e., one streptavidin molecule carries two fluorescent labels. The remaining two binding sites of the streptavidin molecule can capture a biotinylated molecule (e.g., a protein). By mixing labeled streptavidin and the biotinylated molecule in a 1:1 ratio, it can be achieved that in average only one biotinylated molecule is attached to the streptavidin-linker complex. However, the stoichiometry can also be exploited in favor for biotinylated dimeric proteins (e.g., biotinylated stimulator of interferon genes (STING), divalent streptavidin) where the functional dimer can be labeled with one streptavidin-linker complex. In the case of divalent biotin-binding proteins, the ratio between divalent biotin-binding protein and modified linker would be adjusted in a way that in average one of the two free binding sites on the divalent biotin-binding protein is occupied (e.g., by supplying the vial with 2 nM streptavidin and 2 nM linker) and the remaining binding site is available for binding events with the biotinylated particle of interest.

The correct stoichiometry can be verified during the labeling process by a spectral shift measurement according to the present invention (e.g., by using the dual-emission configuration), in which streptavidin is titrated against the linker.

While the free linker molecule (i.e. a 12-mer poly-T strand labeled with biotin at its 3′ end and Cy5 at its 5′ end) has a ratio of smaller than 0.8 (see FIG. 29B, free linker) and streptavidin labeled with only one linker molecule has a ratio of approx. 1.05 (see FIG. 29B, one linker), streptavidin labeled with two linker molecules has a higher ratio, i.e. approx. 1.15, corresponding to a peak in the biphasic dose-response curve, which results from the interaction of both fluorescent labels with the remaining two binding sites of the streptavidin molecule (see FIG. 29B, ideal ratio). When the biotinylated molecule is added to the streptavidin-linker complex the ratio decreases. Since all biotin binding sites retain their full activity, biotinylated molecules can be captured with extremely high affinity, resulting in a characteristic kink at the stoichiometry point in the dose-response curve (see FIG. 29C).

According to this exemplary aspect of the present invention, very low final concentrations of kit components and the biotinylated target molecule (e.g., 1 nM streptavidin, 2 nM linker and 1 nM biotinylated molecule) are used. As such, the kit according to the fifth aspect of the present invention is suitable to measure picomolar affinities based on a highly controllable and reproducible labeling process (as compared to the labeling kits known in the art, e.g., Protein His-Tag Labeling Kit RED-tris-NTA 2nd Generation (NanoTemper Technologies) by using the methods of the present invention. In the same context, presently applied kits known in the art (such as the Protein His-Tag Labeling Kit RED-tris-NTA 2nd Generation) have further limitations including buffer limitations, slow labeling binding kinetics, non-compatibility with already biotinylated molecules and in addition the labeling kits known in the art are cost-intensive and labor-intensive.

### General Principles and Illustrative Aspects

In the following, general principles of the present invention will be discussed in further detail and based on illustrative examples or preferred embodiments of the present invention.

Since the fluorescence spectrum of a fluorescent label strongly depends on the microenvironment in which it is located, the same label can show a very different fluorescence, for example depending on to which molecule or particle it is attached to. Particularly, the microenvironment on each protein around the attachment site of the fluorescent label differs with respect to the amino acid residues that might quench the label, collide with the label, transiently interact with the label or lead to stacking.

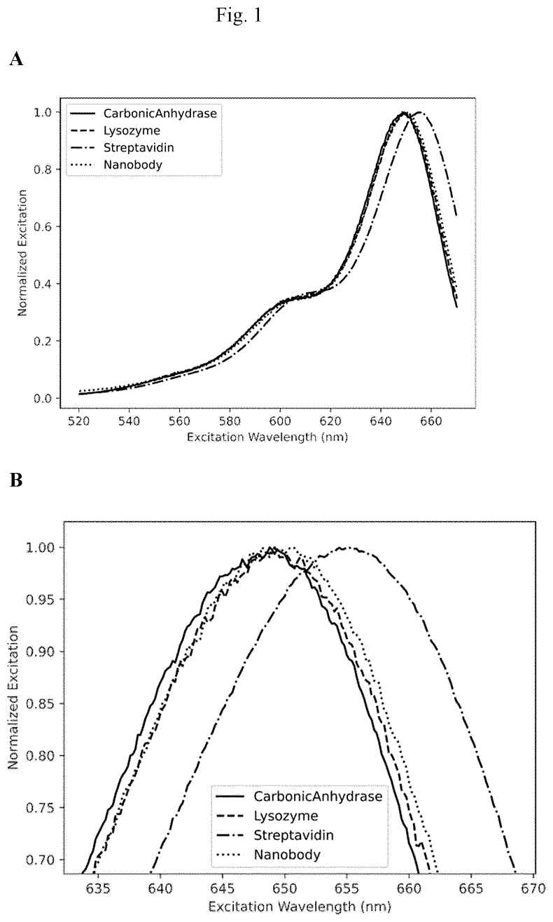

FIG. 1A shows the excitation spectra of four different proteins labeled with an identical fluorescent label (Protein Labeling Kit RED-NHS 2nd Generation (NanoTemper Technologies)). The emission was detected at a wavelength of 690 nm. The excitation was varied between 520 nm and 670 nm. Although being labeled with the same fluorescent label, the maximum emission peak wavelength of the four proteins differs and ranges from approx. 659 nm to approx. 664 nm (FIG. 1B).

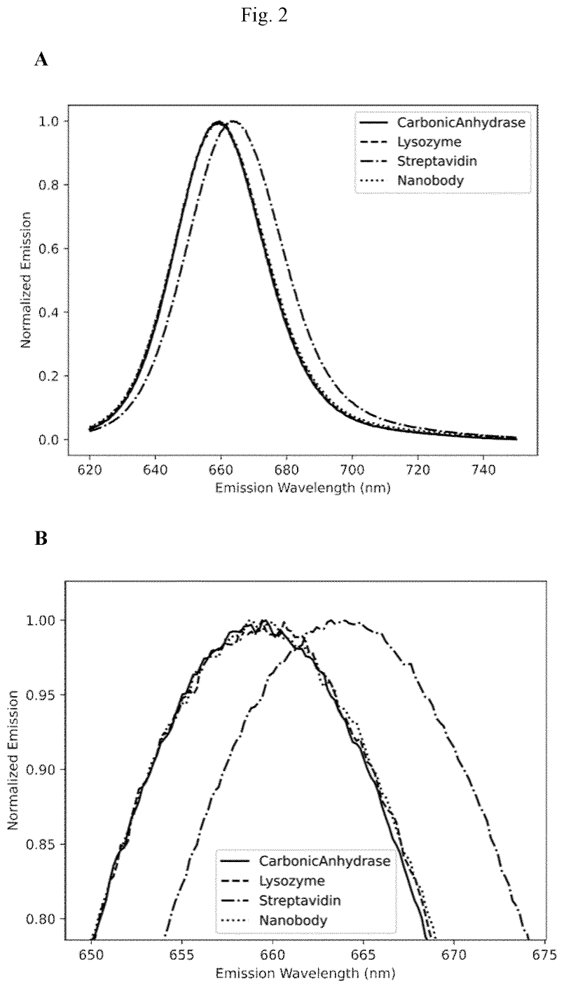

From FIG. 2A it is evident that the same effect/phenomenon was detected when exciting the four different proteins at a constant wavelength (i.e. 605 nm) and recording the emission between 620 nm and 750 nm. Similar to the above, although the proteins were labeled with the same fluorescent label, the maximum emission peak wavelengths ranged from approx. 659 nm to approx. 664 nm (FIG. 2B).

The microenvironment can further change based on the position on said molecule/particle (e.g. to which lysine residue of a protein the labeled is attached to, or whether a label is attached to the 3′ or the 5′ end of a nucleic acid molecule), the conformation of said molecule/particle (e.g. folded or unfolded protein) or the length/composition of the linker between said molecule/particle and the label. The macroenvironment of the fluorescent label also has an impact on its fluorescence spectrum. For example, depending on the location of the particle/molecule (e.g. in an aqueous buffer solution, in a LNP, in a cell) the fluorescence spectrum of said label differs.

Additionally, the fluorescence spectrum of the fluorescent label can also change when the fluorescently labeled particle/molecule is in complex with one or more other molecules (i.e. ligands). For example, ligand proximity (FIG. 3A) and/or conformational changes upon binding of a ligand (FIG. 3B) can lead to a shift (FIG. 3C) and/or broadening or narrowing (FIG. 3D) of the excitation or emission fluorescence spectrum of the fluorescent label. By using the ratiometric characterization method of the present invention it is not only possible to detect changes in the fluorescence intensity, but also the changes in the complete absorption and the emission spectra upon alterations of the fluorescent label's microenvironment.

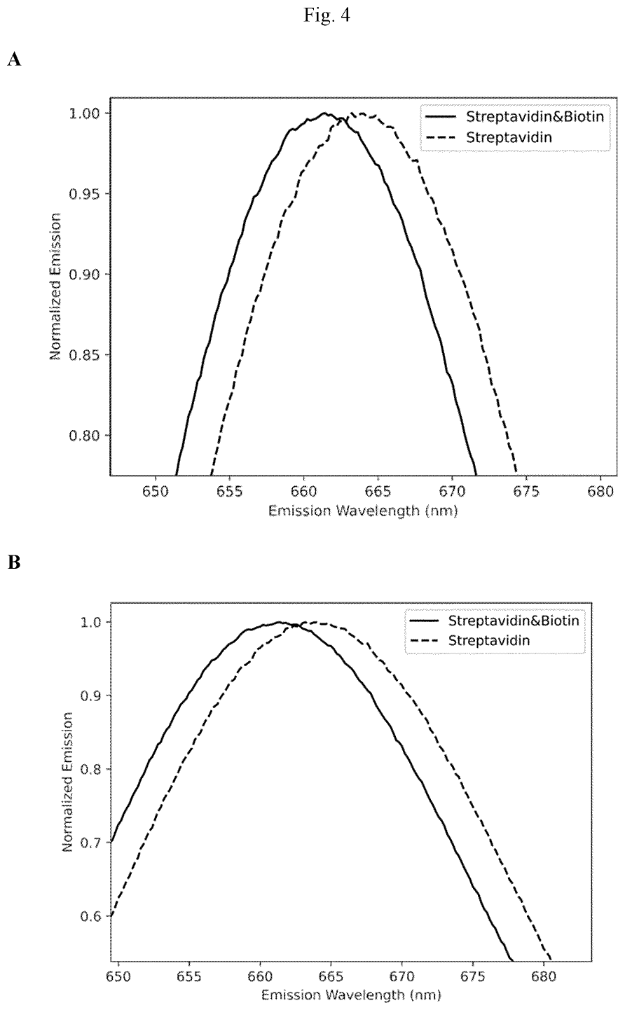

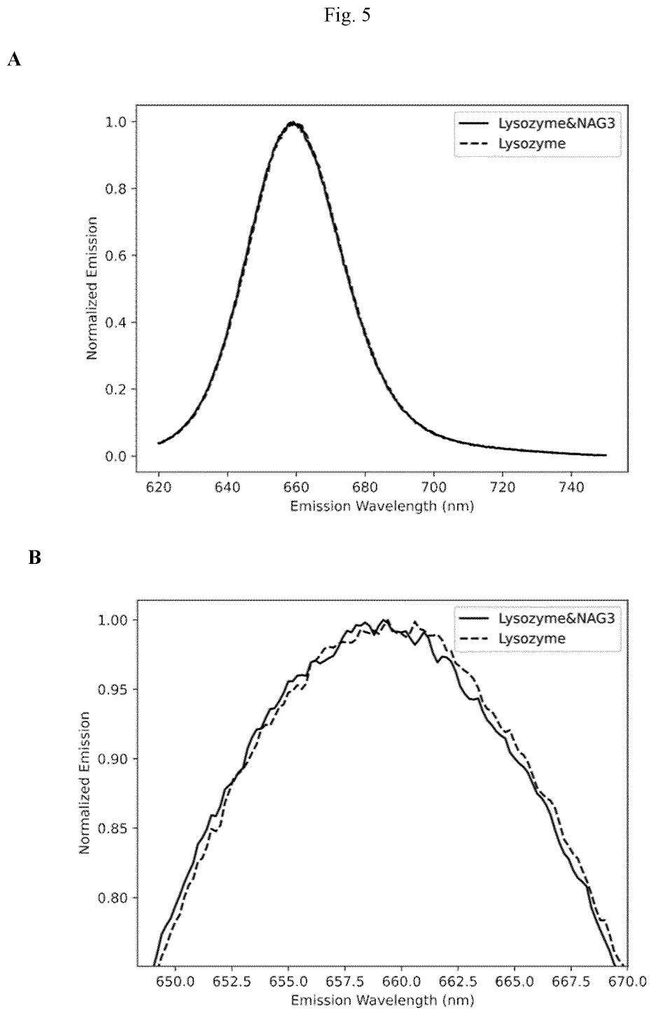

In view of the above, FIG. 4 shows the shift (i.e. 3 nm) of the maximum emission peak wavelength of streptavidin (200 nM) from approx. 664 nm to approx. 661 nm when being in complex with its natural ligand biotin (2 μM) upon excitation at a wavelength of 605 nm. In the same line, FIG. 5 shows a slight shift (i.e. <1 nm) of the maximum emission peak wavelength of lysozyme (100 nM) alone and in complex with the lysozyme inhibitor Tri-N-acetyl-D-glucosamine (NAG3) (80 μM) upon excitation at a wavelength of 585 nm. Shifts of 3 nm and of approx. 500 pm correspond to relative changes in the fluorescence ratio of about 37.3% and 5.5%, respectively (Table 3).

TABLE 3

| | |
|---|---|
| Spectral shift | Ratio change |

| | | |
|---|---|---|
| 3 | nm | 37.3% |
| 2 | nm | 23.6% |
| 1 | nm | 11.2% |
| 500 | pm | 5.5% |
| 100 | pm | 1.1% |
| 50 | pm | 0.5% |

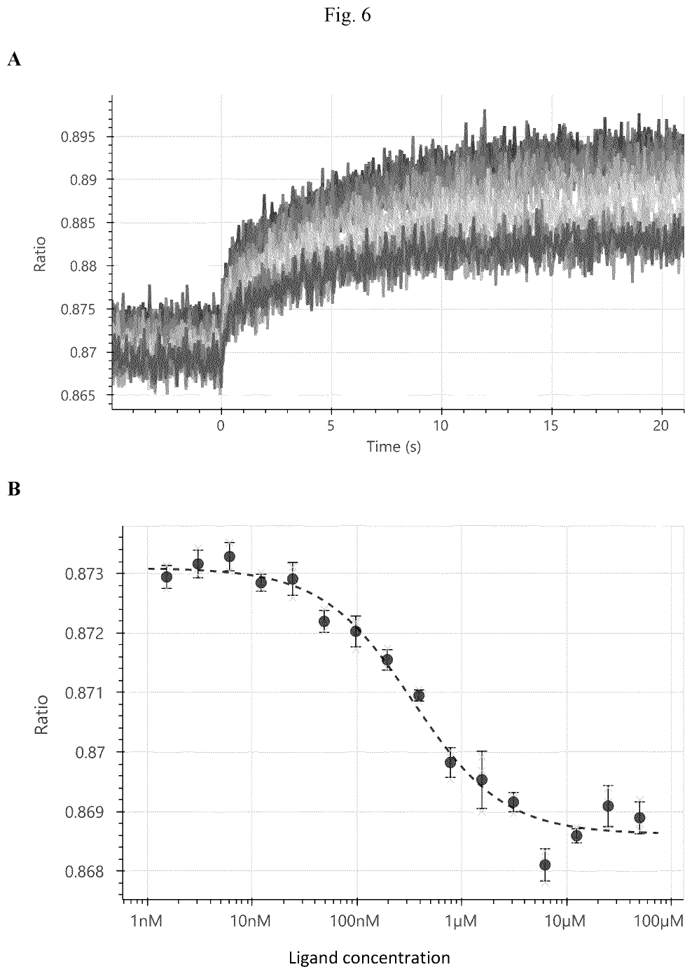

Since, alterations of the excitation and/or emission spectrum of a fluorescent label can be very small (e.g. only a few Å), it is not possible to measure/resolve them with the required precision with the methods/devices known in the art. However, with the methods and the devices of the present invention, even small changes in the fluorescence spectrum can be resolved. For example, wavelength shifts of only 50 pm resulting in a change of the fluorescent ratio of 0.5% (Table 3) as in the case of binding between Carbonic anhydrase and furosemide (FIG. 6A) can be easily measured/detected and a sigmoidal dose-response curve can be obtained (FIG. 6B).

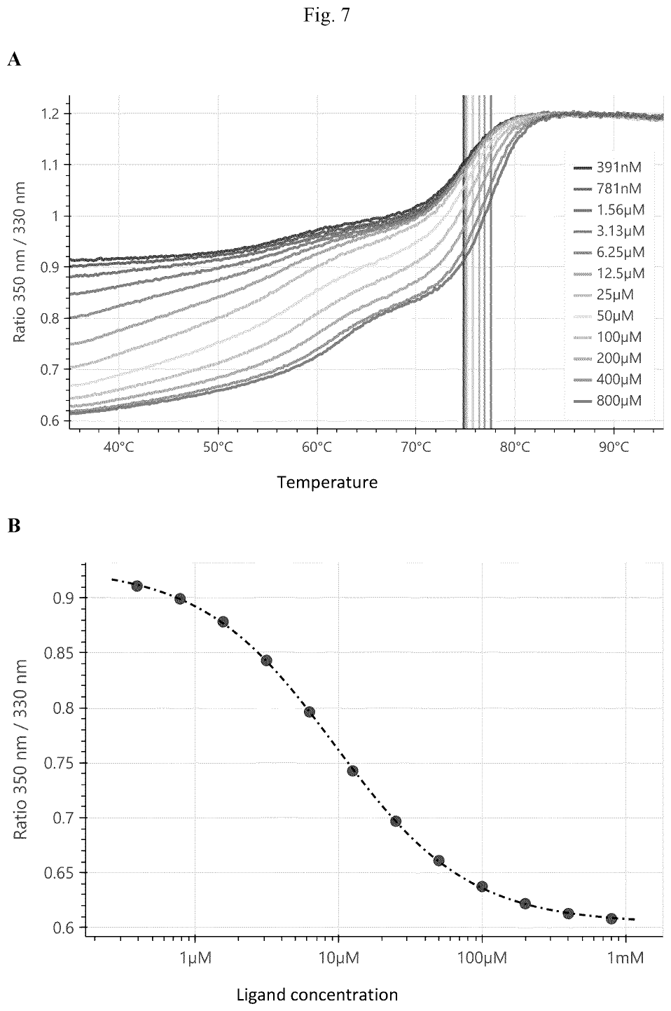

The alterations in the fluorescence spectrum are independent of the nature of the fluorescent label, i.e. they equally appear in intrinsic as well as extrinsic fluorescent labels. However, the extend of the alterations in the fluorescence spectrum may be increased for certain classes of labels. FIG. 7A shows the use of the fluorescence ratio measurement of tryptophan fluorescence during a thermal melting ramp from 35° C. to 95° C. for the characterization of the denaturation as well as the binding affinity between native lysozyme and its inhibitor Tri-N-acetyl-D-glucosamine (NAG3). Very high concentrations of NAG3 lead to a thermal stabilization i.e. a thermal shift of lysozyme. While this shift cannot be used to obtain the dissociation constant (K_{d}), a sigmoidal dose-response curve revealing the K_{d} (here: at 35° C.) can be obtained when plotting the initial ratio at 35° C. against the concentration of NAG3 (FIG. 7B).

FIGS. 8A to 8F shows different exemplary embodiments of measurement devices according to the present invention. In general, a device according to the present invention preferably comprises a sample holder for holding a sample of fluorescently labeled particles in solution under a plurality of conditions. As mentioned above, the sample holder of the present invention can be a capillary without being restricted to such a capillary. Also, other means for holding the sample, such as a multi-well or chip may be used.

FIG. 8A to 8F show examples for arrangements of optical elements which help to direct the light for exiting to the sample and for detecting the fluorescence emission(s) from the sample, wherein the sample itself is not shown in the figures. Preferably, a sample container, e.g., a capillary is located below lens 1. Said lens 1 is preferably an aspheric lens or a lens system with a plurality of lenses, in the following also called objective.

The device of the present invention also comprises at least a means for exciting the fluorescently labeled particles at a first wavelength. For instance, a light source 8 for providing the excitation light may be provided. As discussed above, the present invention is not restricted to a single excitation light source. Alternatively, to the use of one light source, a second excitation light source 16 or even more additional light sources (not shown) may be provided (particularly for a “dual-excitation” mode). The ratiometric analysis of the present invention can be obtained by either using a “dual-excitation” configuration or a “dual-emission” configuration. For the “dual-emission” configuration it is preferred to provide at least one light source. For the “dual-excitation” configuration it is preferred to provide two or even more light sources. A person skilled in the art, however, further understands that a plurality of light sources may be provided for the “dual-emission” configuration. In this case, however, it would be sufficient if one of these light sources is used for the excitation.

The first excitation light source 8 is preferably at least one of the group consisting of a laser, fibre laser, diode-laser, LED, HXP, Halogen, LED-Array, HBO. The same is true for the second excitation light source 16.

Preferably a first light separation element 7, e.g. a dichroic mirror, is used to direct the excited light to the sample and preferably to separate fluorescence excitation light from the fluorescent emission light. Additional optical elements for directing the excitation light to the sample may be provided, e.g., a lens system 9, e.g. to determine beam properties of the excitation light source (e.g. one, two or more lenses). Moreover, also an excitation filter 10 may be provided to filter the excited light, e.g. band pass/long pass. A person skilled in the art would understand which kinds of filters are preferred for the different light sources. Again, similar optical elements may be provided with regard to the second excitation light source 16. For instance, as shown in FIGS. 8C, 8D, 8E and 8F, a lens system 17 may be provided, e.g., to determine beam properties of the second excitation light source 16. Moreover, a further light separation element 18, e.g. a dichroic mirror, may be used, e.g. to combine the light from the two different excitation light sources 8 and 16.

The device of the present invention also comprises means for detecting the fluorescence emission intensity of the fluorescently labeled particles. For the “dual-emission” configuration it is preferred to provide a means for detecting two different wavelength and for the “dual-excitation” configuration it would be sufficient if the means for detecting is configured to detect only a single wavelength or single wavelength range. According to the present invention it is preferred to provide at least one light detector for each wavelength or wavelength range. For instance, for the “dual-excitation” configuration it would be sufficient to provide a single light detector 14 (see e.g., FIGS. 8C and 8D). For the “dual-emission” configuration it is preferred to provide two separate light detectors 14 and 15, as shown in FIGS. 8A, 8B, 8E and 8F). A person skilled in the art, however, further understands that two light detectors may be provided for the “dual-excitation” configuration. In this case, however, it would be sufficient if only one of these light detectors 14 and 15 is used for the emission detection. The first and/or the second light detector may be a light detector of the group consisting of PMT, siPM, APD, CCD or CMOS camera.

Again, for separating the emission light to the first and second light detector 14 and 15, additional optical elements may be provided, such as a light separation element 11. For instance, FIG. 8B shows a preferred configuration for a “single-excitation” “dual-emission” configuration with a excitation light source 8 and two light detectors 14 and 15. The emission light to the individual detectors is separated by the light separation element 11, e.g. a dichroic mirror. Additional filters 12 and 13 upstream of the two detectors 14 and 15 may be provided to the define the different emission wavelength, e.g., second and third wavelength, wherein the second wavelength is shorter, and the third wavelength is longer than an emission maximum of the fluorescence emission of the fluorescently labeled particles under the first conditions. Said emission filters 12 and 13 may be selected from any suitable kind of filter elements, e.g., band pass or long pass filter.

As shown in FIGS. 8A, 8C and 8E, a hot mirror 2 may be additionally provided, which is preferably used for directing IR light from an IR laser 3 to the sample. For instance, the hot mirror 2 may provide a high IR-reflection and preferably a visible light transmission >80%. The IR light source 3 is preferably at least one IR laser, preferably with an emission wavelength of e.g., 1455 nm, 1480 nm, 1550 nm, and/or 980 nm. Moreover, the power of the IR laser preferably is preferably between 0.01 W-10 W. In order to direct the IR light to the sample, further optical elements, such as a laser fibre 4 (single mode or multimode), laser fibre coupler 5 (with or without collimator) and/or a beam shaping module 6, e.g., to determine laser beam diameter and focusing (e.g., lens system comprising one, two or more lenses) may be used (see FIGS. 8A, 8C and 8E). The input of IR light, however, is only optional for additional temperature dependent measurements or additional measurements.

The device of the present invention also comprises a means for calculating a ratio between said fluorescence intensities at the second and the third wavelength, wherein said third wavelength is different from said second wavelength. Said means is preferably provided by a processor or a circuit comprising at least one processor.

### EXAMPLES

### Example 1

The following example illustrates the difference in using ratiometric characterization methods known in the art in comparison to the use of the ratiometric characterization method according to the present invention in order to obtain a dose-response curve between two molecules. Therefore, a sample containing fluorescently labeled DNA aptamer and adenosine monophosphate (AMP) was measured with a commercially available fluorescence spectrophotometer and with the dual-emission configuration of the present invention.

### Sample Preparation

A 14-point 1-to-1 dilution series of unlabeled AMP was prepared. The DNA aptamer was fluorescently labeled with Cy5 and added in equal amounts to the AMP dilution series to obtain a final sample concentration of 20 nM. The highest concentration of AMP corresponded to 5 mM. For measurements with a standard fluorescence microplate-reader (CLARIOstar, BMG Labtech) 95 11.1 of the sample were loaded into a micro-well plate. For measurements with the dual-emission configuration of the present invention 5 to 10 μl of the samples were loaded into polymer-coated borosilicate glass capillaries (Monolith NT.115 Premium Capillaries, MO-K025, NanoTemper Technologies).

### Measurement

For the microplate-reader measurements, the sample was excited at a wavelength of 590 nm and the emission was first detected at a wavelength of 628 nm to 652 nm. Then, the sample was again excited at the 590 nm wavelength and the emission was detected at a wavelength of 665 nm to 725 nm. Three subsequent fluorescence intensity measurements of the dilution series were run. For the ratiometric measurement according to the present invention, each sample was excited at a wavelength of 591 nm. The fluorescence traces were simultaneously measured at a second wavelength between 628 nm to 653 nm and a third wavelength between 665 nm to 727 nm.

### Ratiometric Data Analysis

In order to characterize the interaction with the fluorescence microplate-reader, the ratio between the fluorescence intensities per well was calculated manually by using a commercially available calculation tool (Microsoft Excel). The fluorescence detected at the higher wavelength was divided by the fluorescence detected at the lower wavelength. The sigmoidal dose-response curve provided in FIG. 10A starts at a ratio value of approx. 0.95 and ends at a ratio value of approx. 0.90. The midpoint, which corresponds to the dissociation constant (K_{d}) of the interaction, lies at approx. 20-30 μM. However, the signal-to-noise ratio (S/N) of the interaction is very low and the deviation between the replicates is very large.

In contrast, by measuring the same samples according to method of the present invention the dose-response curve provided in FIG. 10B shows an improved signal-to-noise ratio (S/N) and clearly reveals the K_{d} of the interaction at 39.3 μM. When using lower sample concentrations, i.e. 250 pM, for the ratiometric measurement according to the present invention, the signal-to-noise ratio (S/N) is still very good (19.8) and a K_{d} of the interaction can be easily determined (FIG. 10C).

In summary, this example proves that for the ratiometric characterization method according to the present invention approx. 1000× less sample still gives better data compared to the measurement with the plate reader. For measurements with commercially available microplate-readers the noise is more than 10× higher than the signal amplitude that needs to be measured.

### Example 2

The following example describes the use of the ratiometric characterization method according to the present invention in order to obtain a dose-response curve between two molecules based on the dual-emission configuration. Therefore, a sample containing fluorescently labeled DNA aptamer and AMP was excited at a first wavelength and the emitted fluorescence intensity was measured at a second and a third wavelength.

### Sample Preparation

A 12-point 1-to-1 dilution series of unlabeled AMP was prepared. The DNA aptamer was fluorescently labeled with Cy5 and added in equal amounts to the AMP dilution series to obtain a final sample concentration of 20 nM. The highest concentration of AMP corresponded to 2 mM. The samples were then loaded into polymer-coated borosilicate glass capillaries (Monolith NT.115 Premium Capillaries, MO-K025, NanoTemper Technologies).

### Measurement

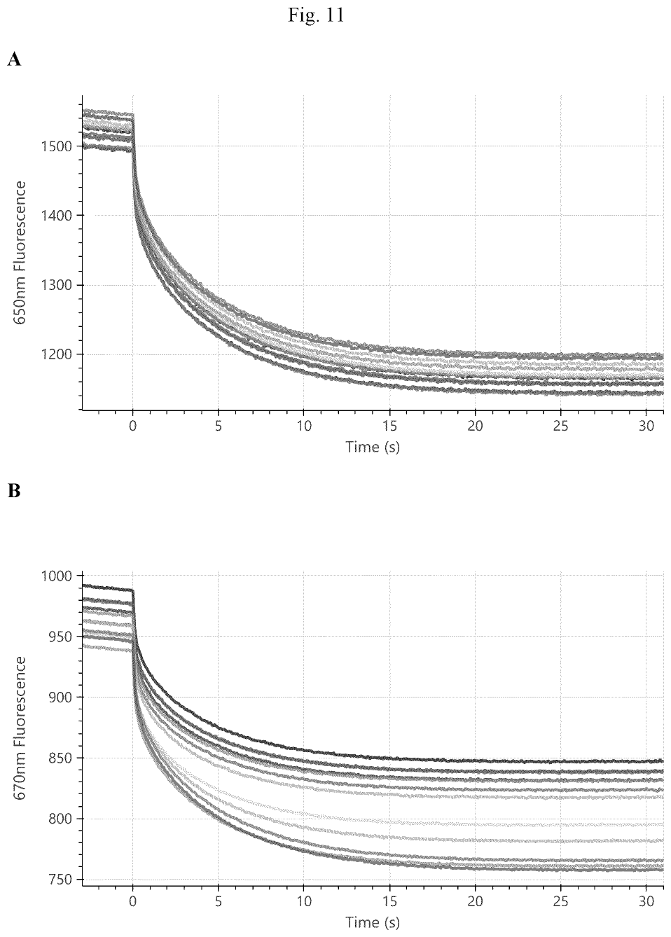

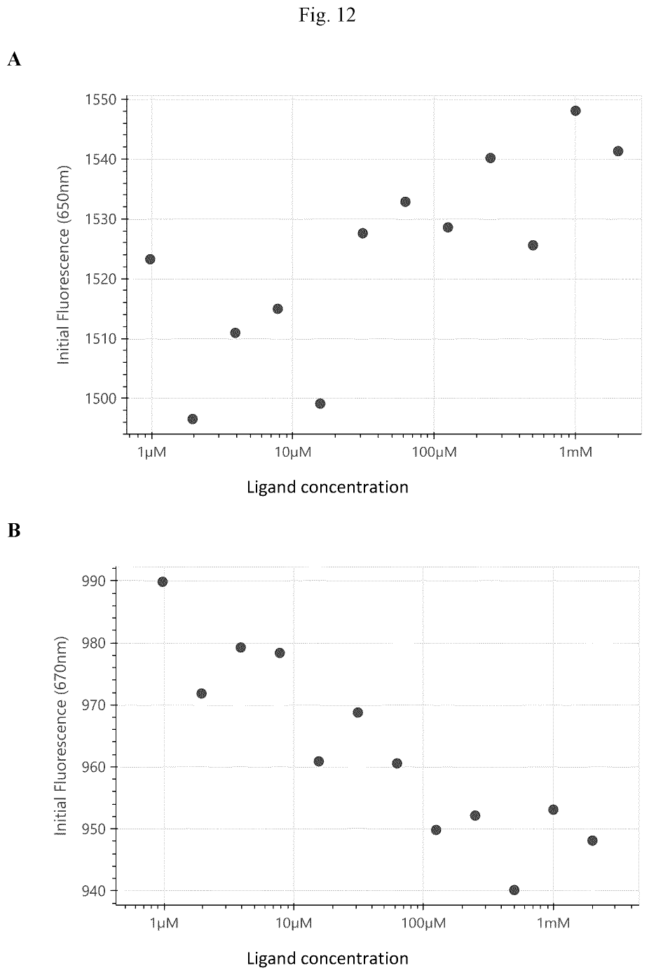

Each sample was excited at a wavelength of 591 nm. At the timepoint 0 s, an IR laser was switched on. The response of the fluorescence intensity was simultaneously measured for 31 s. The fluorescence traces recorded between 628 nm to 653 nm (“650nm”) are provided in FIG. 11A. The fluorescence traces recorded between 665 nm to 727 nm (“670 nm”) are provided in FIG. 11B. Since the initially recorded fluorescence intensities showed a large variation, no sigmoidal dose-response curve for affinity determination could be obtained. Consequently, no binding information could be extracted from the initial fluorescence of neither of the two emission wavelengths provided in FIG. 12.

### Ratiometric Data Analysis

For the ratiometric analysis (i.e. obtaining the ratios of the fluorescence traces), pointwise division of the fluorescence traces at 670 nm and 650 nm was performed. The obtained ratio traces can be analyzed either before (FIG. 13A, Phase 1) or after the IR laser is turned on (FIG. 13A, Phase 2 or Phase 3). By ratiometrically analyzing the data recorded before the IR laser was turned on (FIG. 13A, Phase 1), a dose-response curve with a signal-to-noise ratio (S/N) higher than 300, yielding the K_{d} between both molecules at the sample temperature, i.e. room temperature, was obtained (FIG. 13B).

By performing the ratiometric analysis after turning on the IR laser (FIG. 13A, Phase 2 or Phase 3), K_{d} values at higher temperatures can be obtained. This approach is especially recommended if the amplitude at room temperature is very small and is expected to increase at higher temperatures.

### Determination of Thermodynamic Parameters of the Interaction

In order to obtain a K_{d}-over time curve of the interaction, “vertical slices” in a time interval of 200 ms of the ratio traces provided in FIG. 13A were taken and the K_{d} was determined of the dose-response curves for each of these slices (FIG. 14A). Since the temperature change over time was known from a calibration measurement, i.e. the sample tray was heated in a controlled way and the fluorescence of a reference dye was measured, and the interaction was equilibrating on a faster time-scale then the occurring temperature change, a K_{d}-over-temperature relationship could be obtained. By performing Van't Hoff analysis:

$$ln ( {K}_{d} ) = \frac{Δ {H}^{0}}{RT} - \frac{Δ {S}^{0}}{R}$$

the binding enthalpy (ΔH) and binding entropy (ΔS) (FIG. 14B) of the interaction were determined. For this interaction it was found that AMP binds predominantly enthalpically (ΔH<0).

### Example 3

The following example describes the use of the ratiometric characterization method according to the present invention in order to obtain a dose-response curve between two molecules based on the dual-excitation configuration. Therefore, a sample containing fluorescently labeled DNA aptamer and AMP was excited at a first and a second wavelength and the emitted fluorescence intensity was measured at a third wavelength.

### Sample Preparation

A 16-point 1-to-1 dilution series of unlabeled AMP was prepared. The DNA aptamer was fluorescently labeled with Cy3 and added in equal amounts to the AMP dilution series to obtain a final sample concentration of 250 nM. The highest concentration of AMP corresponded to 12.5 mM. The samples were then loaded into polymer-coated borosilicate glass capillaries (Monolith NT.115 Premium Capillaries, MO-K025, NanoTemper Technologies).

### Measurement

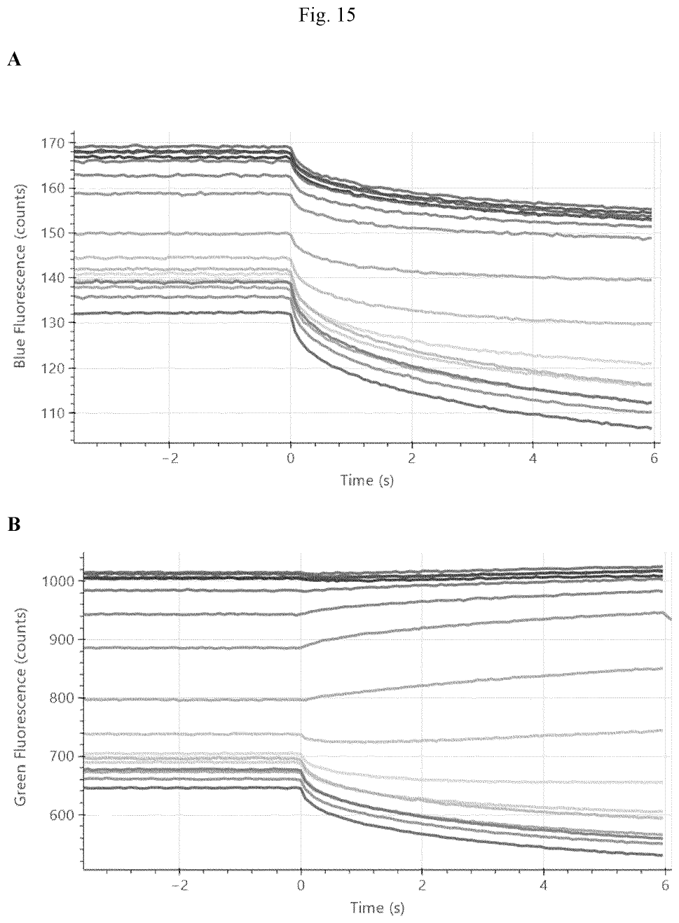

Each sample was excited at a first wavelength of 480 nm (“blue” LED). At the timepoint 0 s, an IR laser was switched on. The response of the fluorescence intensity was detected from 590 nm to 680 nm for 6 s. In the following, each sample was excited at a second wavelength of 540 nm (“green” LED). At the timepoint 0 s, an IR laser was switched on. The response of the fluorescence intensity was recorded by a detector from 590 nm to 680 nm for 6 s. The fluorescence traces obtained by exciting the sample with the “blue” LED are provided FIG. 15A. The fluorescence traces obtained by exciting the sample with the “green” LED are provided in FIG. 15B.

### Ratiometric Data Analysis

For the ratiometric analysis (i.e. obtaining the ratios of the fluorescence traces), pointwise division of the fluorescence traces obtained via excitation with the green LED by the ones obtained via excitation with the blue LED was performed. The obtained ratio traces can be analyzed either before (FIG. 16A Phase 1) or after the IR laser is turned on (FIG. 16A Phase 2 or Phase 3). By ratiometrically analyzing the data recorded before the IR laser was turned on (FIG. 16A, Phase 1), a dose-response curve with a signal-to-noise ratio (S/N) of approx. 80 yielding the K_{d} between both molecules at sample temperature, i.e. room temperature was obtained (FIG. 16B). By ratiometrically analyzing the data recorded after the IR laser was turned on (FIG. 16A, Phase 3), a dose-response curve with a signal-to-noise ratio (S/N) greater than 130, yielding the K_{d} between both molecules was obtained (FIG. 16C). Compared to the analysis at room temperature, an improved signal-to-noise ratio was obtained when the ratios were analyzed at higher temperatures.

### Example 4

The following example describes the use of the ratiometric characterization method according to the present invention in order to obtain a dose-response curve between two molecules based on the dual-emission configuration. Therefore, a sample containing fluorescently labeled streptavidin and its naturally occurring ligand biotin was excited at a first wavelength and the emitted fluorescence intensity was measured at a second and a third wavelength.

### Sample Preparation

A 12-point 1-to-1 dilution series of unlabeled biotin was prepared. Streptavidin was fluorescently labeled with the Protein Labeling Kit RED-NHS 2nd Generation (NanoTemper Technologies) and added in equal amounts to the biotin dilution series to obtain a final sample concentration of 20 nM. The highest concentration of biotin corresponded to 500 nM. The samples were then loaded into polymer-coated borosilicate glass capillaries (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”).

### Ratiometric Data Analysis

For the ratiometric analysis (i.e. obtaining the ratios of the fluorescence traces), pointwise division of “670 nm” by “650 nm” was performed. As evident from the dose-response curve shown in FIG. 17, the ratio changed from a value of approx. 2.2 to approx. 1.2 when streptavidin is unbound or in complex with biotin, respectively. Since the target concentration is much higher than the K_{d}, a characteristic kink at the stoichiometry point at a concentration of 80 nM biotin can be observed.

### Example 5

The following example describes the use of the ratiometric characterization method according to the present invention in order to obtain a dose-response curve between two molecules based on the dual-emission configuration. Therefore, a sample containing fluorescently labeled bovine carbonic anhydrase II and acetazolamide was excited at a first wavelength and the emitted fluorescence intensity was measured at a second and a third wavelength.

### Sample Preparation

A 15-point 1-to-1 dilution series of unlabeled acetazolamide was prepared. Bovine carbonic anhydrase II was fluorescently labeled with the Protein Labeling Kit RED-NHS 2nd Generation (NanoTemper Technologies) and added in equal amounts to the dilution series of acetazolamide to obtain a final sample concentration of 20 nM. The highest concentration of acetazolamide corresponded to 2.5 μM. The samples were then loaded into polymer-coated borosilicate glass capillaries (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”).

### Ratiometric Data Analysis

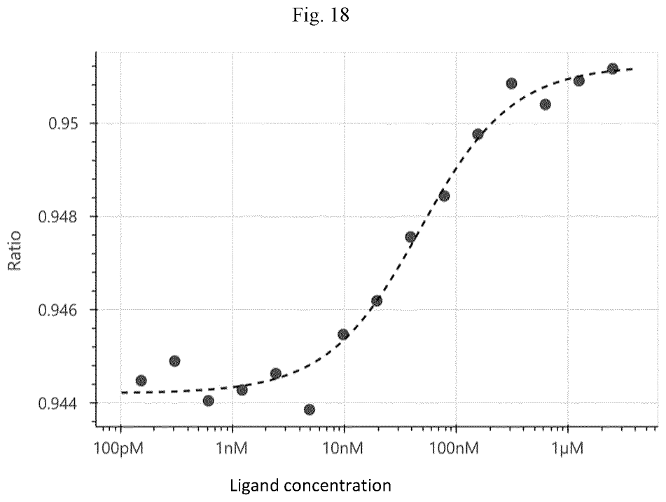

For the ratiometric analysis (i.e. obtaining the ratios of the fluorescence traces), pointwise division of “670 nm” by “650 nm” was performed. In the dose-response curve provided in FIG. 18, the ratio changed from a value of approx. 0.944 to approx. 0.951 and hence, about approx. 0.7%. The obtained dose-response curve had a signal-to-noise ratio (S/N) greater than 30. This reveals that even small changes in the ratio can be measured with the ratiometric characterization method of the present invention.

### Example 6

The following example describes the use of the ratiometric characterization method according to the present invention in order to obtain a dose-response curve between three molecules based on the dual-emission configuration. Therefore, a sample containing fluorescently labeled monovalent streptavidin, biotinylated protein L and the antibody Herceptin was excited at a first wavelength and the emitted fluorescence intensity was measured at a second and a third wavelength.

### Sample Preparation

A 16-point 1-to-1 dilution series of unlabeled antibody Herceptin was prepared. Monovalent streptavidin was fluorescently labeled with the Protein Labeling Kit RED-NHS 2nd Generation (NanoTemper Technologies) and 20 nM of it were mixed with an equal volume of 4 nM biotinylated protein L. The mix was then added in equal amounts to the Herceptin dilution series to obtain a final sample concentration of 5 nM labeled monovalent streptavidin and 1 nM biotinylated protein L in the assay. The highest concentration of Herceptin corresponded to 1 μM. The samples were then loaded into polymer-coated borosilicate glass capillaries (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”).

### Ratiometric Data Analysis

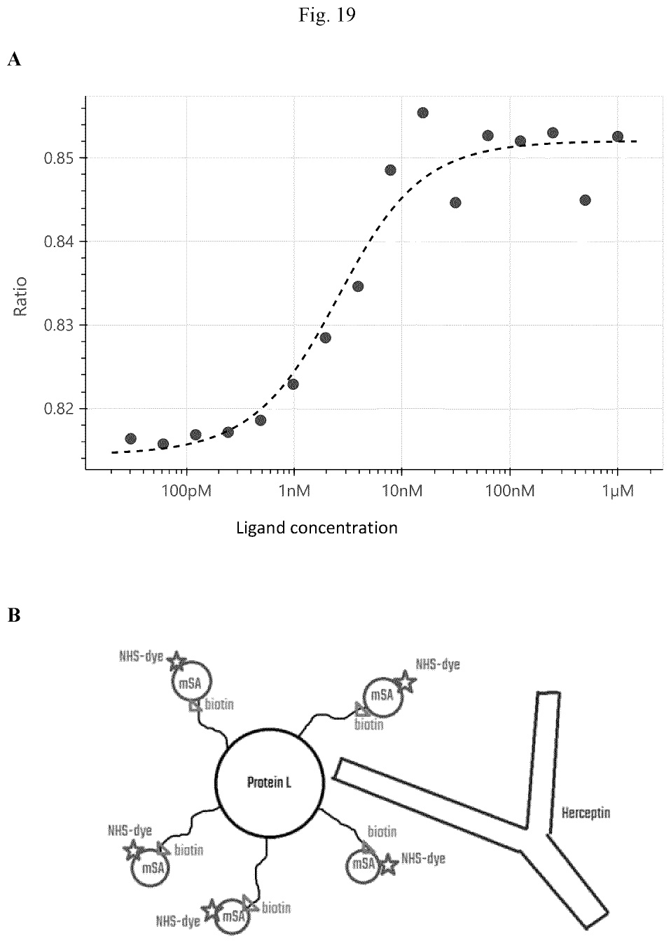

For the ratiometric analysis (i.e. obtaining the ratios of the fluorescence traces), pointwise division of “670 nm” by “650 nm” was performed. The dose-response curve provided in FIG. 19A, reveals that the method of the present invention enables measurements of ternary complexes, in which the labeling is performed indirectly via a labeled third molecule (FIG. 19B) such as labeled streptavidin for the measurement of the interaction between a biotinylated protein and a ligand.

### Example 7

The following example describes the use of the ratiometric characterization method according to the present invention in order to obtain a dose-response curve between a small molecule and a protein that is biotinylated (i.e. a biotin molecule is covalently attached to it) based on the dual-emission configuration. For fluorescent labeling, the biotinylated protein was mixed with the protein streptavidin (SA) and a short nucleic acid modified with biotin at its 3′-end and the fluorophore Cy5 at its 5′ -end (bDNA). SA is a homo-tetramer that has an extraordinarily high affinity for biotin (also known as vitamin B7). It is used extensively in molecular biology and bio-nanotechnology due to the streptavidin-biotin complex's resistance to organic solvents, denaturants (e.g., guanidinium chloride), detergents (e.g., SDS, Triton), proteolytic enzymes, and extremes of temperature and pH.

In the example, a sample containing maltose binding protein (MBP) was fluorescently labeled by following this approach. MBP was then mixed with the small molecule maltose and excited at a first wavelength and the emitted fluorescence intensity was measured at a second and a third wavelength.

### Sample Preparation

Streptavidin was prepared at a stock concentration of 1 mg/mL (around 19 μM) and then diluted to 4 nM in phosphate-buffered saline. bDNA (a 12-mer oligo-dT sequence with a Cy5 molecule attached at its 5′ end and a biotin molecule attached at its 3′ end) was chemically synthesized and ordered from a DNA vendor. A 100 μM stock solution was prepared and then diluted in double-distilled water (ddH₂O) to a final concentration of 8 nM. SA and bDNA were then mixed in a 1:1 volume ratio to obtain a 4 nM SA, 8 nM bDNA solution (1:2 stoichiometry). Through this step, SA became fluorescently labeled by binding to the Cy5-labeled biotinylated bDNA.

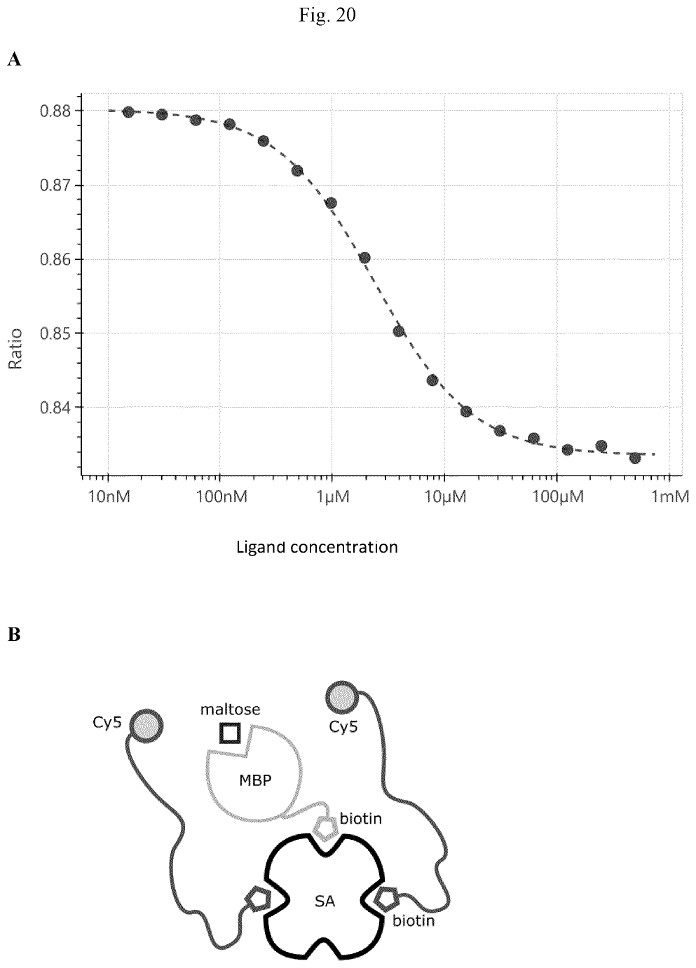

Next, 100 μl of 100 nM biotinylated MBP were mixed with 100 ρl of the 4 nM SA, 8 nM bDNA solution to obtain 200 μl of a 2 nM SA, 4 nM bDNA, 50 nM MBP solution. Since SA is a tetrameric protein, in average two of its four binding sites for biotin are unoccupied and can bind the biotinylated MBP in order to create a bDNA-SA-MBP complex, i.e. fluorescently labeled MBP (FIG. 20A).

Next, a 16-point 1-to-1 dilution series of unlabeled maltose was prepared. The fluorescently labeled MBP was added in equal amounts to the maltose dilution series to obtain a final target concentration of 1 nM SA, 2 nM bDNA and 25 nM MBP. The highest concentration of maltose corresponded to 500 μM. The samples were then loaded into polymer-coated borosilicate glass capillaries (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”). Each capillary was measured for a time of 3 seconds.

### Ratiometric Data Analysis

For the ratiometric analysis (i.e. obtaining the ratios of the fluorescence traces), pointwise division of “670 nm” by “650 nm” was performed. The dose-response curve provided in FIG. 20B, reveals that the method of the present invention enables measurements of quaternary complexes, in which the labeling is performed indirectly via an unlabeled third and a labeled fourth molecule, such as unlabeled SA and labeled biotinylated single-stranded DNA oligomers.

### Example 8

The following example describes the use of the ratiometric characterization method according to the present invention in order to obtain a dose-response curve between two molecules based on the dual-emission configuration. Therefore, a sample containing fluorescently labeled therapeutic antibody CR3022, Cov-19 (“SARS CoV-2”) Spike protein and the protein Angiotensin-converting enzyme 2 (ACE2) was excited at a first wavelength and the emitted fluorescence intensity was measured at a second and a third wavelength.

### Sample Preparation

A 14-point 1-to-1 dilution series of unlabeled protein ACE2 was prepared. Therapeutic antibody CR3022 was fluorescently labeled with the Protein Labeling Kit RED-NHS 2nd Generation (NanoTemper Technologies) and 10 nM of it were mixed with an equal volume of 80 nM Cov-19 Spike protein. The mix was then added in equal amounts to the ACE2 dilution series to obtain a final sample concentration of 5 nM labeled CR3022 and 20 nM of Spike protein. The highest concentration of ACE2 corresponded to 250 nM. The samples were then loaded into polymer-coated borosilicate glass capillaries (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”).

### Ratiometric Data Analysis

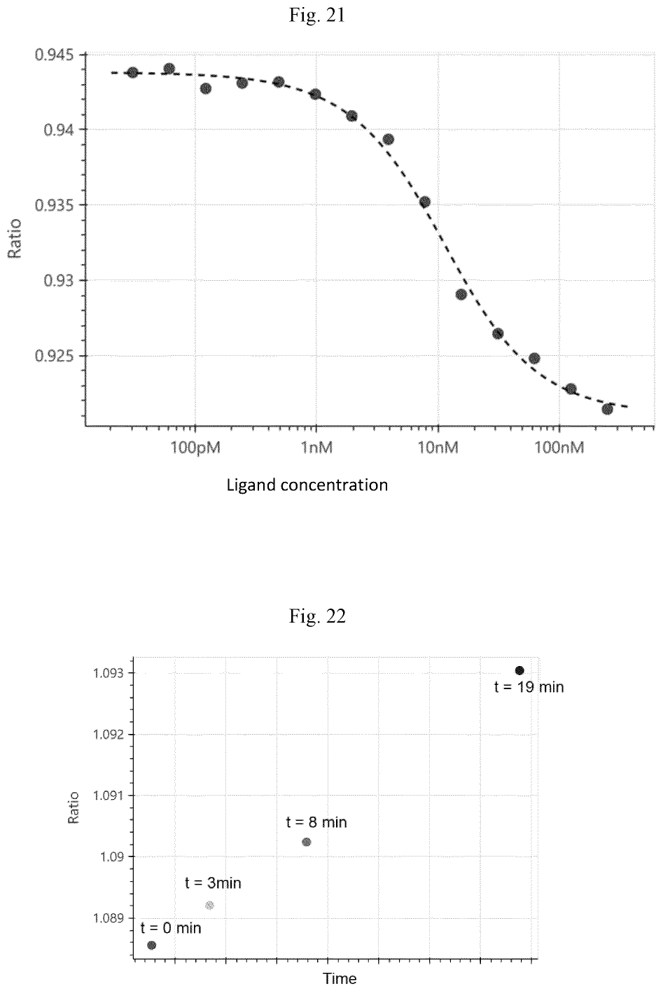

For the ratiometric analysis (i.e. obtaining the ratios of the fluorescence traces), pointwise division of “670 nm” by “650 nm” was performed. The dose-response curve provided in FIG. 21, reveals that the method of the present invention enables measurements of ternary complexes, in which the labeling is performed indirectly via a labeled third molecule such as a labeled antibody.

### Example 9

The following example describes the use of the ratiometric characterization method according to the present invention in order to characterize the conformational state of a protein based on the dual-emission configuration. Therefore, a sample containing fluorescently labeled protein was excited at a first wavelength and the emitted fluorescence intensity was measured at a second and a third wavelength.

### Sample Preparation

Mitogen-activated protein kinase 14 (p38-α) was fluorescently labeled with the Protein Labeling Kit RED-NHS 2nd Generation (NanoTemper Technologies). The labeled protein was then diluted to a concentration of 20 nM and loaded into a polymer-coated borosilicate glass capillary (Monolith NT.115 Premium Capillaries, MO-K025, NanoTemper Technologies).

### Measurement

The sample was excited at a wavelength of 591 nm. The fluorescence trace was simultaneously measured at a second wavelength between 628 nm to 653 nm (“650 nm”) and a third wavelength between 665 nm to 727 nm (“670 nm”). Measurements were performed directly after dilution (t=0 min) and again after 3, 8 and 19 minutes inside the capillary.

### Ratiometric Data Analysis

For the ratiometric analysis (i.e. obtaining the ratios of the fluorescence traces), pointwise division of “670 nm” by “650 nm” was performed. When plotting the fluorescence ratio over the start time of the measurement, provided in FIG. 22, it is revealed that the ratio is not constant over the time period of the four measurements but increases linearly over time. This increase of the ratio indicates that the labeled protein is not stable at room temperature but gradually denatures.

### Example 10

The following example describes the method of the present invention in order to measure fast binding kinetics based on the dual-emission configuration. Therefore, a sample containing fluorescently labeled DNA aptamer for adenosine and the small molecule AMP and a sample containing two 11-mer complementary DNA strands where one DNA strand was fluorescently labeled with Cy5 were excited at a first wavelength. The emitted fluorescence intensities for said samples were measured at a second and a third wavelength.

### Sample Preparation

For the aptamer, a 12-point 1-to-1 dilution series of unlabeled AMP was prepared. The DNA aptamer was fluorescently labeled with Cy5 and added in equal amounts to the AMP dilution series to obtain a final sample concentration of 20 nM. The highest concentration of AMP corresponded to 2 mM. The samples were then loaded into polymer-coated borosilicate glass capillaries (Monolith NT.115 Premium Capillaries, MO-K025, NanoTemper Technologies).

For the DNA hybridization, a 16-point 1-to-1 dilution series of the unlabeled 11-mer (sequence: 5′ CCT GAA GTC C 3′) was prepared. The complementary 11-mer (sequence: 5′ GGA CTT CAG G 3′) was fluorescently labeled at its 5′ end with Cy5 and added in equal amounts to the dilution series to obtain a final sample concentration of 10 nM. The highest concentration of the unlabeled 11-mer corresponded to 100 μM. The samples were then loaded into polymer-coated b orosilicate glass capillaries (Monolith NT.115 Premium Capillaries, MO-K025, NanoTemper Technologies).

### Measurement

Each sample was excited at a wavelength of 591 nm. At the timepoint 0 s, an IR laser was switched on. The response of the fluorescence intensity was simultaneously measured for 6 s (aptamer), respectively 21 s (DNA hybridization). The fluorescence traces were measured at a second wavelength between 628 nm to 653 nm (“650 nm”) and a third wavelength between 665 nm to 727 nm (“670 nm”).

From above it is evident that information on the binding interaction (e.g. K_{d}) can be derived from any “vertical” slice in time along the ratiometric traces, when the method of the present invention is used in combination with fast IR laser heating. A new type of curve, which can be described as K_{d}-over-time curve can be generated. From this curve not only information on the thermodynamics but also on the binding kinetics of the interaction can be determined. In particular, if the equilibration kinetics of an interaction is slower than the heating with the IR laser, the K_{d}-over-time curve will show a characteristic lag.

### Ratiometric Data Analysis

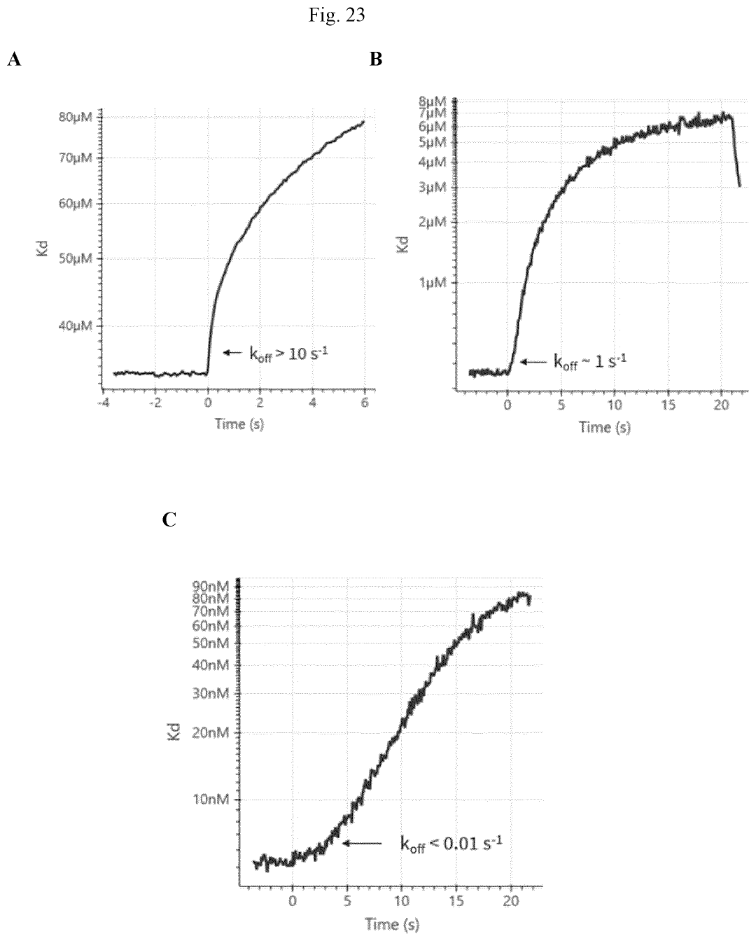

For the ratiometric analysis (i.e. obtaining the ratios of the fluorescence traces), pointwise division of “670 nm” by “650 nm” was performed. The K_{d}-over-time curves provided in FIG. 23 reveal three different interactions with different dissociation constants K_{d} and binding kinetics. For the interaction between the Cy5-labeled DNA aptamer and AMP, a K_{d} of approx. 30 μM was determined and a k_{off} greater than 10 s⁻¹ was estimated (FIG. 23A). For the interaction, i.e. the DNA hybridization, between the two 11-mer complementary DNA strands measured at 32° C., a K_{d} of approx. 500 nM was determined and a koff of approx. 1 s⁻¹ was estimated (FIG. 23B). Measurements of above mentioned two 11-mer complementary DNA strands at 22° C., revealed a K_{d} of approx. 5 nM and a k_{off} smaller than 0.01 s⁻¹ (FIG. 23C).

Detailed information on the K_{d}-over-time measurements between 22° C. and 32° C. for the DNA hybridization described above is provided in FIG. 24A. The y-axis shows the fold increase of K_{d} throughout the measurement. For interactions with slower kinetics, the K_{d}-over-time curve does not follow the temperature change instantly, but rather shows a distinct lag, whereby the lag is the larger, the slower the interaction kinetics are. Analyzing this lag therefore can provide valuable information on the binding kinetics of an interaction. Even if exact values of k_{off} and kₒₙ cannot be obtained, the ability to compare ligands and identify ligands that dissociate faster is already a tremendous benefit of this method.

Furthermore, assuming that after 20 s of IR laser heating the equilibrium is reinstated at the new, higher temperature, a van't Hoff analysis at the two different temperatures, i.e. 22° C. as the initial sample temperature and approx. 32° C. as the temperature after IR laser heating (determined from a calibration experiment as described above), the enthalpy as well as entropy of the interaction were obtained (see FIG. 24B and FIG. 24C). The obtained thermodynamic parameters are similar to the ones obtained from a classical van't Hoff analysis by adjusting the sample tray temperature to multiple different temperatures (e.g. 22° C., 24° C., 26° C., 28° C., 30° C., 32° C.) and measuring the K_{d} at each of them (see FIG. 24D and FIG. 24E).

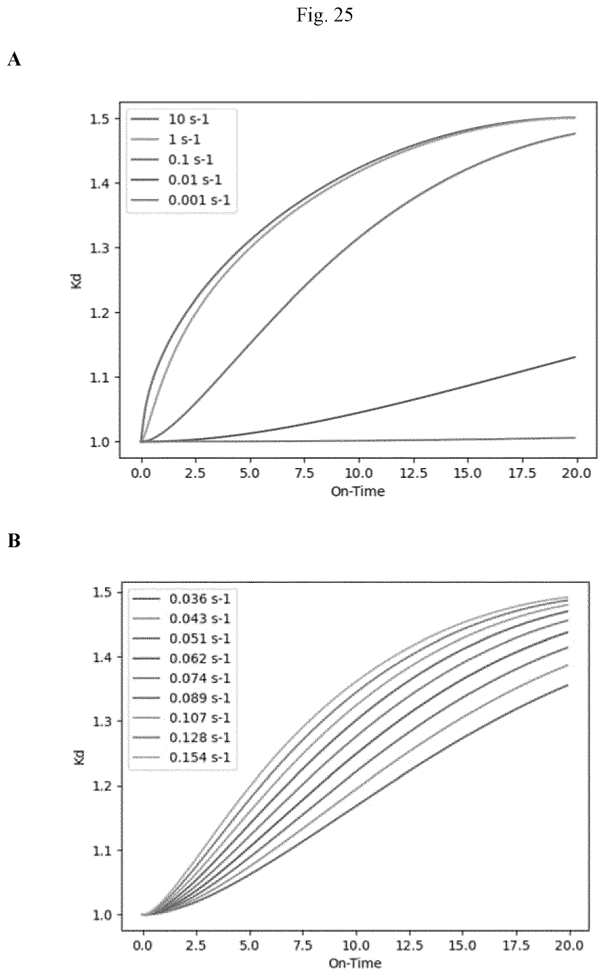

Simulation data on K_{d}-over-time curves for different dissociation rates is provided in FIG. 25. The simulated K_{d}-over-time curves provided in FIG. 25A reveal that dissociation rates between 10 s⁻¹ and 0.001 s⁻¹ can be resolved by performing the method of the present invention in combination with 20 s of IR laser heating. The simulated K_{d}-over-time curves provided in FIG. 25B reveal that even small differences between 0.036 s⁻¹ and 0.154 s⁻¹ can be resolved.

### Example 11

The following example describes the method of the present invention in order to measure slow binding kinetics based on the dual-emission configuration. Therefore, a sample containing a fluorescently labeled nanobody and Cov-19 spike RBD protein was excited at a first wavelength and the emitted fluorescence intensity was measured at a second and a third wavelength.

### Sample Preparation

A nanobody against Cov-19 Spike protein was fluorescently labeled with the Protein Labeling Kit RED-NHS 2nd Generation (NanoTemper Technologies). 2 nM of the fluorescently labeled nanobody were rapidly mixed with six different concentrations of Cov-19 Spike RBD protein ranging from (20 nM to 625 pM). The samples were then loaded into polymer-coated borosilicate glass capillary (Monolith NT.115 Premium Capillaries, MO-K025, NanoTemper Technologies).

### Measurement

Each sample was excited at a wavelength of 591 nm. The fluorescence ratio was simultaneously measured at a second wavelength between 628 nm to 653 nm (“650 nm”) and a third wavelength between 665 nm to 727 nm (“670 nm”). No IR laser was turned on in this example as the samples were measured repeatedly and a temperature change could have influenced the binding kinetics.

### Ratiometric Data Analysis

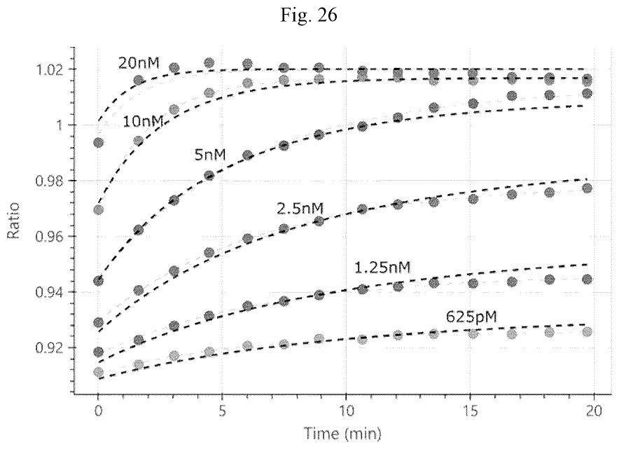

For the ratiometric analysis (i.e. obtaining the ratios of the fluorescence traces), pointwise division of “670 nm” by “650 nm” was performed. The ratio-over-time curve provided in FIG. 26, reveals that the method according to the present invention enables to follow slow binding kinetics when using such a “mix-and-measure” approach and the binding kinetics is slower than the time required to prepare the samples and start the measurement. By applying a global fitting model kₒₙ=6.9×10⁵ WM⁻¹s⁻¹, k_{off}=2.9×10⁻⁴ s⁻¹ and K_{d}=415 pM of the interaction between said nanobody and Cov-19 spike RBD protein were determined.

### Example 12

The following example describes the use of the ratiometric characterization method according to the present invention in order to localize fluorescently labeled particles based on the dual-emission configuration (FIG. 27). Therefore, a sample containing fluorescently labeled mRNA was excited at a first wavelength and the emitted fluorescence intensity was measured at a second and a third wavelength.

### Sample Preparation

mRNA was fluorescently labeled with Atto647N fluorescent dye and incorporated into lipid nano particles (LNPs). Duplicates of these mRNA containing LNP preparations were exposed to different kinds of stress i.e. adding 0.25% of the detergent polysorbate 20 (Tween-20), being boiled at 90° C. for 10 min, being vortexed for 1 min or being centrifuged at 14,000 rpm for 20 min. Untreated mRNA containing LNPs were used as a control. The samples were then loaded into polymer-coated borosilicate glass capillary (Monolith NT.115 Premium Capillaries, MO-K025, NanoTemper Technologies).

### Measurement

Each sample was excited at a wavelength of 591 nm. The fluorescence traces were simultaneously measured at a second wavelength between 628 nm to 653 nm (“650 nm”) and a third wavelength between 665 nm to 727 nm (“670 nm”).

For measurements to determine only the absolute fluorescence ratio, each capillary was measured for a time of 3 seconds. For measurements to determine the aggregation state of the LNPs, measurements with IR laser were conducted (laser on-time of 60 seconds).

From previously performed control measurements provided, it is evident that the ratiometric fluorescence signal of fluorescently labeled mRNA with Atto647N located within the LNPs in the dual-emission configuration of this invention equals to approx. 2.1. In contrast, when all fluorescently labeled mRNA molecules are located outside of the LNPs the ratiometric fluorescence signal equals to approx. 1.2.

### Ratiometric Data Analysis

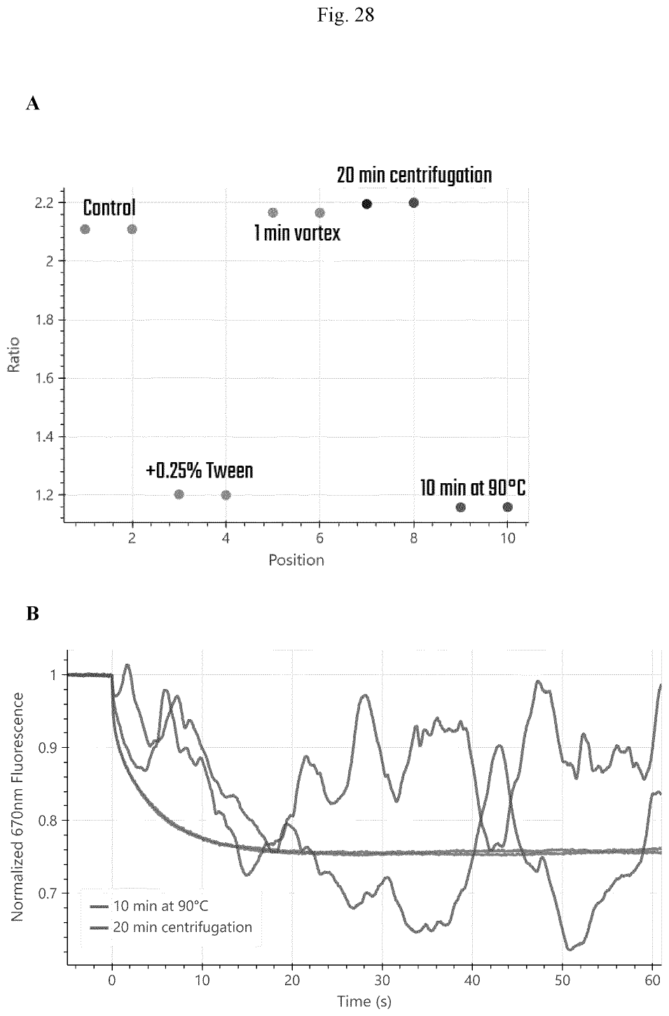

For the ratiometric analysis (i.e. obtaining the ratios of the fluorescence traces), pointwise division of “670 nm” by “650 nm” was performed. For the untreated control, i.e. all fluorescently labeled mRNA molecules are located within the LNPs, the ratiometric fluorescence signal equals to 2.1 (FIG. 28A, Control). By adding high concentrations of detergents, the lipid membrane of the LNPs gets ruptured/damaged and hence, the fluorescently labeled mRNA molecules are no longer incorporated within the LNPs (FIG. 28A, +0.25% Tween). In this case, the ratiometric fluorescence signal equals to 1.2. As evident from a ratio of approx. 2.1, the fluorescently labeled mRNA is still located within the vortexed or centrifugated LNP preparations (FIG. 28A, 1 min vortex, 20 min centrifugation). However, when analyzing the “bumpy” fluorescence traces obtained after the IR laser was turned on (FIG. 28B), said treatments lead to the aggregation of the LNP preparations. In contrast, the ratio of 1.2 obtained when boiling the LNP preparations at 90° C. for 10 min indicated that the fluorescently labeled mRNA was no longer incorporated within the LNPs (FIG. 28A, 10 min at 90° C.). This was further validated by analyzing the fluorescence traces after the IR laser was turned on. Since, fluorescence traces without bumps were observed after boiling, it was confirmed that the fluorescently labeled mRNA molecules had left the (potentially still aggregated) LNPs.

As evident from the above, the ratiometric characterization method of the present invention enables to determine the localization of fluorescently labeled mRNA.

### LIST OF REFERENCE SIGNS

1: Lens (for example an aspheric lens), or lens system, or an objective
2: Hot mirror, high IR-reflection, visible light transmission >80%
3: IR laser (e.g. 1455 nm, 1480 nm, 1550 nm, 980 nm, 0.01 W-10 W) or laser for positioning
4: Laser fibre (single mode or multimode)
5: Laser fibre coupler w/o collimator
6: Beam shaping module to determine laser beam diameter and focusing (e.g. lens system comprising one, two or more lenses)
7: First light separation element (e.g. dichroic mirror) to separate fluorescence excitation from emission
8: First excitation light source (e.g. laser, fibre laser, diode-laser, LED, HXP, Halogen, LED-Array, HBO)
9: Lens system to determine beam properties of the excitation light source (e.g. one, two or more lenses)
10: Excitation filter (e.g. band pass/long pass)
11: Second light separation element (e.g. dichroic mirror) to split emission in lower and higher wave-length component
12: First emission filter (e.g. band pass/long pass)
13: Second emission filter (e.g. band pass/long pass)
14: First light detector (e.g. PMT, siPM, APD, CCD or CMOS camera)
15: Second light detector (e.g. PMT, siPM, APD, CCD or CMOS camera)
16: Second excitation light source (e.g. laser, fibre laser, diode-laser, LED, HXP, Halogen, LED-Array, HBO)
17: Lens system to determine beam properties of the excitation light source
18: Third light separation element (e.g. dichroic mirror) to combine two different excitation light sources

### CITED NON PATENT LITERATURE

[NPL1] Sindrewicz, P., Li, X., Yates, E. A., Turnbull, J. E., Lian, L. Y., & Yu, L. G. (2019). Intrinsic tryptophan fluorescence spectroscopy reliably determines galectin-ligand interactions. Scientific reports, 9(1), 1-12.
[NPL2] Mayer-Wrangowski, S. C., & Rauh, D. (2015). Monitoring ligand-induced conformational changes for the identification of estrogen receptor agonists and antagonists. Angewandte Chemie International Edition, 54(14), 4379-4382.
[NPL3] Chen, H. J., Chew, C. Y., Chang, E. H., Tu, Y. W., Wei, L. Y., Wu, B. H., . . . & Tan, K. T. (2018). S-cis diene conformation: a new bathochromic shift strategy for near-infrared fluorescence switchable dye and the imaging applications. Journal of the American Chemical Society, 140(15), 5224-5234.
[NPL4] Niu, W., Wei, Z., Jia, J., Shuang, S., Dong, C., & Yun, K. (2018). A ratiometric emission NIR-fluorescent probe for sensing and imaging pH changes in live cells. Dyes and Pigments, 152, 155-160.
[NPLS] Pauli, J., Grabolle, M., Brehm, R., Spieles, M., Hamann, F. M., Wenzel, M., . . . & Resch-Genger, U. (2011). Suitable labels for molecular imaging—influence of dye structure and hydrophilicity on the spectroscopic properties of IgG conjugates. Bioconjugate chemistry, 22(7), 1298-1308.
[NPL6] Harroun, S. G., Lauzon, D., Ebert, M. C., Desrosiers, A., Wang, X., & Vallée-Bélisle, A. (2022). Monitoring protein conformational changes using fluorescent nanoantennas. Nature methods, 19(1), 71-80.

All patent and non-patent documents cited herein are hereby incorporated by reference in their entirety.

1. A method for the characterization of fluorescently labeled particles in solution by analyzing alterations in the fluorescence spectrum of the fluorescently labeled particles, comprising the steps:
a) providing a sample of the fluorescently labeled particles in a solution under first conditions,
b) exciting the fluorescently labeled particles at a first wavelength,
c) detecting the fluorescence emission intensity of the fluorescently labeled particles at a second and a third wavelength,
d) calculating the ratio between said fluorescence intensities at the second and the third wavelength,
wherein said third wavelength is different from said second wavelength,
e1) repeating steps b) to d) for said sample of the fluorescently labeled particles under second conditions, or
e2) repeating the steps a) to d) for a second sample of the fluorescently labeled particles under second conditions,
wherein said second conditions are different from said first conditions,
f) characterizing the fluorescently labeled particles based on the calculated ratios obtained for the different conditions,
wherein the second and third wavelength are detected simultaneously, and wherein the second wavelength is shorter, and the third wavelength is longer than an emission maximum of the fluorescence emission of the fluorescently labeled particles under the first conditions.

2-20. (canceled)
